(19) United States
(12) Patent Application Publication
(10) Pub. No.: US 2023/0036685 A1
(43) Pub. Date: Feb. 2, 2023

(54) Novel Vectors and Uses Thereof

(71) Applicants: John Douglas BURKE, Rockville, MD (US); Fabio CANDOTTI, Chardonne (CH)

(72) Inventors: John Douglas BURKE, Rockville, MD (US); Fabio CANDOTTI, Chardonne (CH)

(21) Appl. No.: 17/754,211

(22) PCT Filed: Sep. 30, 2020
(86) PCT No.: PCT/US2020/053353

§ 371 Date: Mar. 27, 2022

Related U.S. Application Data
(60) Provisional application No. 62/907,718, filed on Sep. 29, 2019.
(60) Provisional application No. 63/038,884, filed on Jun. 14, 2020.

(51) Int. Cl. C12N 15/86; C12N 9/22; C12N 15/11; C12N 15/90

(52) U.S. Cl. CPC C12N 15/86; C12N 9/22; C12N 15/11; C12N 15/907; C12N 2310/20; C12N 2800/80; C12N 2740/15043; C12N 2740/15052; C12N 2740/15022; C12N 2740/15042

(57) ABSTRACT

Provided is a viral particle comprising a genomic RNA suitable for delivering a polynucleotide sequence of interest (SOI) into a cell and/or a subject. The genomic RNA comprises, from 5′ to 3′: (a) the SOI replacing the upstream R, (b) a U5, (c) a primer binding site (PBS), (d) an encapsidation signal (Psi), (e) polypurine tract(s) (PPT), and (f) a U3. The SOI preferably takes the form of single-stranded DNA or a DNA-RNA hybrid after initiation of reverse transcription. Additionally provided are polynucleotides, vectors, cells, components, compositions, kits, methods and uses of the viral particle.

### STATEMENT OF GOVERNMENT SUPPORT

This invention was made with governmental support under Grant No. ZIAHG000122 awarded by the National Institutes of Health. The Government has certain rights in the invention.

### BACKGROUND

Viruses of the taxonomic family Retroviridae include the orthoretroviruses, e.g., gammaretroviruses (RV) and lentiviruses (LV), and the spumaviruses. Vectors based on the RV and LV have been used extensively for gene transfer, owing both to the efficiency with which they transduce target cells and the stability of the transfer. The Retroviridae have genomes comprised of two copies of plus-sense single-stranded RNA. On entry of the virus into target cells, the genomic RNA is reverse transcribed to double-stranded DNA, which is then integrated into host cell chromosomal DNA to form a provirus. The proviral DNA then serves as a template for transcription and translation of virally encoded genes, expression of which allows for the production of new virions.

Genomes of the Retroviridae consist of the following components at minimum, in order 5′ to 3′: R-U5-PBS-Psi-Gag-Pol-Env-PPT-U3-R, with more complex viruses encoding additional accessory genes that assist in the viral replicative cycle. The genes gag, pol and env encode viral structural proteins, as well as enzymes necessary to complete the viral life cycle, e.g., reverse transcriptase and integrase. U3, R, and U5 comprise the viral terminal redundancies, or long terminal repeats (LTRs), that characterize the members of this family, with the U3 and U5 regions being copied to the opposite LTR during reverse transcription.

Normally, reverse transcription is initiated at a cellular tRNA annealed to the primer binding site (PBS) of the virus. The polymerase domain of the reverse transcriptase (RT) extends the minus strand DNA using the viral genomic RNA as template, while the RNase H domain degrades the latter essentially simultaneously. When the RT reaches the 5′ end of the RNA, the complex undergoes a nucleocapsid and RT-mediated “strong stop jump” to the homologous R region at the 3′ end of the viral genome, to which the nascent minus strand DNA anneals. RT is then able to complete minus strand synthesis of the remainder of the genome. As the first strand is being completed, second strand synthesis is initiated. The entire process normally yields a double-stranded DNA reverse transcript.

For reasons of safety, traditional gene transfer vectors based on the Retroviridae were engineered to be replication defective. This was accomplished by splitting the essential viral elements onto separate vector-encoding constructs. Most commonly, the gag and pol genes would be inserted into one expression vector, while env would be cloned into another. A third viral construct would encode the sequence of interest (SOI), flanked by all the elements required to be in cis with the SOL e.g., LTRs, to enable its vector-mediated transfer. All three constructs would be introduced/transfected into cultured cells permissive for viral production. After sufficient time had passed to allow these “producer” cells to generate adequate amounts of vector, usually 48-72 hours, the culture supernatant would be harvested and the vector concentrated and purified, depending on application.

Despite the considerable efforts made to improve on vector safety detailed above, vectors based on the Retroviridae remain problematic. Perhaps of greatest concern, the DNA reverse transcripts they produce integrate more or less randomly into the target cell genome. Even in the context of integrase deficiency, the DNA may still integrate spontaneously into the genomic DNA. These integrations are mutagenic and have been confirmed to induce target cells to become malignant. Extensive work has therefore been undertaken to develop vector systems that allow for manipulations of target cells but minimize the likelihood of undesired integrations and insertional mutagenesis.

Over the last several decades, numerous modifications of viral components and processes have been investigated extensively for myriad purposes and potential applications. Notably, Applicant and others found previously that the sequence of the R region of the vector 5′ LTR is dispensable for vector production and reasonable maintenance of titer (Adam, Osborne & Miller, 1995; Cheslock, Anderson, Hwang, Pathak & Hu, 2000). Also noteworthy was the discovery by others that mutations of the RNase H domain of MuLV RT yield reverse transcripts that retain the viral genomic RNA, producing RNA-DNA hybrids in transduced cells, and do so without drastically affecting vector packaging and titer (Tanese & Goff, 1988; Blain & Goff, 1995). A final notable finding by other groups was that mutations of the viral RT impeded the strong stop jump of reverse transcription, again without greatly affecting vector titer (Herzig, Voronin, Kucherenko & Hizi, 2015).

There exists in the art a variety of methods for delivering the polynucleotides discussed above to mammalian cells. Physiochemical means are perhaps the most common, while viral vectors are also used, albeit less frequently. A number of issues with both delivery modes have prevented their widescale adoption in the clinic. Regarding the physiochemical systems, low efficiencies of delivery, particularly in vivo, have greatly limited their utility; and degradation of the polynucleotides by target cell nucleases has proven a considerable obstacle to their successful use.

Thus, there remains a need for vectors and methods suitable for delivering a polynucleotide to a cell or a subject efficiently and safely. This disclosure satisfies these needs and provides related advantages as well.

### SUMMARY OF THE DISCLOSURE

In one aspect, the disclosure described herein relates to retrovirus- and/or lentivirus-like vectors that encode DNA and/or RNA SOIs that have been cloned to replace the R region of the vector upstream, or 5′, LTR. The downstream R region, or any other sequence homologous to the SOI, is absent; hence, the vectors lack complete LTRs and the terminal redundancy that exist in traditional retro- and lentivirus-based vectors. In one embodiment, the vectors are designed to arrest at the strong stop of reverse transcription such that the SOIs only undergo minus strand DNA synthesis, thereby producing reverse transcripts of other than double-stranded DNA. Additionally or alternatively, the SOIs are cloned in such a way, i.e., in place of the upstream R regions, to minimize viral sequences flanking them, such that the SOIs ultimately appear at one or both ends of the reverse transcripts. Depending upon application, the vectors may be packaged with mutant viral enzymatic components that are defective in their roles in reverse transcription.

Additionally or alternatively, provided are retro- and lentiviral-like vectors, defined in part by their lack of the terminal redundancy, or complete LTRs, present in typical vectors of this sort.

In one embodiment, the vectors are designed to arrest reverse transcription of heterologous SOIs at the strong stop. In a further embodiment, the aim of the vector is to produce RNA-DNA hybrid and/or single-stranded DNA reverse transcripts of the SOIs. In one embodiment, these are generated by cloning the SOIs into the 5′ vector LTR, into the upstream most region of the genomic RNA. In some embodiments of hybrid-producing vectors, the sequence of interest is cloned into the upstream R site of the vector, with no homologous sequence present in the downstream R site, and is packaged with an RT mutated in its RNase H domain such that degradation of the vector genomic RNA is precluded, e.g., Y586F, D524N, Δ5E, ΔC, and/or H7 in RV or E478Q in LV, thereby yielding a reverse transcript comprised of, or alternatively consisted essentially of, or yet further consisted of the SOI in RNA-DNA hybrid form. In some embodiments of the single-stranded DNA-producing vector, the sequence of interest is again cloned into the upstream R site, but here the RT does not have a modification of its RNase H domain, instead preferably having a point mutation that precludes strand transfer, e.g., L92P and/or F61A in LV and Y598V in RV. Alternatively, the nucleocapsid (NC) of the vector may be mutated to preclude the strand transfer. Again, there is no homologous sequence in the downstream LTR of the vector, so, without to be bound by the theory, in addition to being unable to make the strong stop jump due to mutation of the RT or NC, there is no place for the nascent minus strand DNA to anneal, thereby leaving the reverse transcript/SOI in single-stranded DNA form.

In one embodiment, the RNA provided comprises or consists essentially of, or yet further consists of, optionally from 5′ to 3′: (a) a SOI, (b) a U5, (c) a PBS, (d) an encapsidation signal (Psi), (e) an optional internal ribosome entry site (IRES), (f) an optional coding sequence encoding a protein, (g) polypurine tract sequence(s) (PPT), (h) a U3, and (i) a polyadenylation (pA) signal and polyA tail. In one aspect, the RNA is an isolated and/or engineered RNA. In a further embodiment, the protein encoded by the coding sequence of (f) is a clustered regularly interspaced short palindromic repeats (CRISPR) associated (Cas) enzyme or similar RNA-guided endonuclease. In some embodiments, the SOI comprises, or consists essentially of, or yet further consists of a sense or antisense strand of a donor template polynucleotide. In some embodiments, the SOI comprises or consists essentially of, or yet further consists of a micro RNA, a small interfering RNA (siRNA), or a messenger RNA (mRNA). In some embodiments, the SOI comprises, or consists essentially of, or yet further consists of an antisense strand of: a donor template polynucleotide or an antisense oligonucleotide (ASO). In some embodiments, the SOI comprises, or consists essentially of, or yet further consists of one or more RNA sequences, each of which is complementary to either strand of a double-strand DNA which can be recognized and cut by a restriction enzyme (i.e. either strand of a cloning site). In one embodiment, the SOI comprises, or consists essentially of, or yet further consists of RNA sequence complementary to either strand of a multiple cloning site (MCS). In some embodiments, the SOI comprises, or consists essentially of, or yet further consists of one or more pairs of RNA sequences, each pair of which comprises, or consists essentially of, or yet further consists of two RNA sequences complementary to either strand of a pair of homology arms suitable for use in recombineering (i.e. either strand of a recombineering site). In some embodiments, the RNA lacks an R region. In one embodiment, the RNA comprises a pA signal, optionally an SV40 pA signal.

Also provided is a polynucleotide complementary to or corresponding to or encoding the RNA as disclosed herein. In one aspect, the polynucleotide is an isolated and/or engineered polynucleotide. In some embodiments, the polynucleotide comprises, or consists essentially of, or yet further consists of one or more cloning sites and/or either strand of the one or more cloning sites. In some embodiments, the polynucleotide comprises, or consists essentially of, or yet further consists of one or more recombineering sites and/or either strand of the one or more recombineering sites.

In a further aspect, provided is a vector (such as a viral particle, including but not limited to a retroviral particle or a lentiviral particle) comprising the polynucleotide and/or RNA as disclosed herein. In some embodiments, the vector further comprises one or more of the following: a protein encoded by a gag gene (such as a group-specific antigen precursor polyprotein and/or its processed group-specific antigen polyprotein(s) including but not limited to a nucleocapsid (NC), a capsid protein (CA) or a matrix protein (MA)), a protein encoded by a pol gene (such as a precursor polyprotein encoded by a pol gene and/or its processed polyprotein(s) including but not limited to a reverse transcriptase (RT), an RNase H domain optionally as part of a RT or any other polypeptide, an integrase (IN), or a protease (PR)), a protein encoded by an env gene (such as a precursor polyprotein encoded by an env gene and/or its processed polyprotein(s) including but not limited to a surface envelope protein and a transmembrane envelope protein), an endonuclease, a polynucleotide encoding an endonuclease, a detectable marker, a selection marker, or a lipid bilayer (for example, packaging the polynucleotide and/or RNA therein). In some embodiments, the RNase H domain is a wildtype RNase H domain or a mutant thereof defective in degrading RNA. In some embodiments, the RT is a wildtype RT or a mutant thereof defective in degrading RNA and/or mediating strand transfer. In some embodiments, the IN is a wildtype integrase or a mutant thereof defective in integrating a polynucleotide into a chromosomal DNA. In some embodiments, the NC is a wildtype NC or a mutant thereof defective in mediating strand transfer.

In yet a further aspect, provided is a cell comprising one or more of the following: an RNA as disclosed herein, a polynucleotide as disclosed herein, or a vector (such as a viral particle, including but not limited to a retroviral particle or a lentiviral particle) as disclosed herein.

Additionally provided are methods for producing a vector (such as a viral particle, including but not limited to a retroviral particle or a lentiviral particle) as disclosed herein. In one embodiment, the methods are in the context of packaging constructs encoding vector gag and pol genes mutated so as preclude the strong stop jump of reverse transcription.

Further provided are methods for transducing a cell (such as a eukaryotic cell, and/or a cell in a subject) with a vector (such as a viral particle, including but not limited to a retroviral particle or a lentiviral particle) as disclosed herein, methods for delivering a polynucleotide to a cell, and methods for delivering a polynucleotide to a subject. In some embodiments, the methods comprises or consists essentially of, or yet further consists of mediated entry of the vector into the target cell and optionally transiting of the payload (such as sense and/or antisense strand(s) of SOI) to the cell nucleus. In some embodiments, the vector payload is protected from cellular nucleases by incorporation into, for example, the viral pre-integration complex. In some embodiments, the delivered polynucleotide comprises, or consists essentially of, or yet further consists of the sequence of interest (SOI) or a polynucleotide which is a reverse complement of the SOI or both.

Yet further provided is a composition comprising, or alternatively consisting essentially of, or yet further consisting of an RNA as disclosed herein, a polynucleotide as disclosed herein, and/or a vector (such as a viral particle, including but not limited to a retroviral particle or a lentiviral particle) as disclosed herein, a carrier and an optional guide polynucleotide. The carrier can be suitable for in vitro, in vivo, diagnostic or therapeutic use. Additionally provided is a kit comprising, or alternatively consisting essentially of, or yet further consisting of one or more of: an RNA as disclosed herein, a polynucleotide as disclosed herein, a vector (such as a viral particle, including but not limited to a retroviral particle or a lentiviral particle) as disclosed herein, an expression vector comprising one or more of: a gag gene, a pol gene, or an env gene, an expression vector encoding proteins required for the RNA to be packaged in a particle, a guide polynucleotide, a composition as disclosed herein, and an instruction for use.

The disclosure can comprise, or consist essentially of, or yet further consist of any of the aspect and/or embodiments, alone or in combination.

### BRIEF DESCRIPTION OF THE DRAWINGS

FIG. 1 provides schematic representations of a standard gammaretroviral (RV) or lentiviral (LV) genomic RNA, an exemplified genomic RNA of a viral vector/particle as disclosed herein, and two possible products of reverse transcription of the exemplified genomic RNA, i.e., an RNA-DNA hybrid and a single-stranded DNA. Thick line indicates an RNA sequence, while thin lines represent DNA sequences.

FIGS. 2A to 2B provide a corresponding DNA sequence (FIG. 2A) and a linear schematic representation (FIG. 2B) of the upstream portion of an example polynucleotide, comprising, from 5′ to 3′, a U3 region, a SOI comprising a cloning site but without an R region, a U5 region, a PBS and a Psi.

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

The practice of the present technology will employ, unless otherwise indicated, conventional techniques of tissue culture, immunology, molecular biology, microbiology, cell biology, and recombinant DNA, which are within the skill of the art. See, e.g., Animal Cell Culture (R. I. Freshney, ed. (1987)), Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3rd edition (Cold Spring Harbor Laboratory Press (2002)); and Sohail (ed.) (2004) Gene Silencing by RNA Interference: Technology and Application (CRC Press).

Throughout this disclosure, various publications, patents and published patent specifications are referenced by an identifying citation. The disclosures of these publications, patents and published patent specifications are hereby incorporated by reference into the present disclosure in their entirety to more fully describe the state of the art to which this invention pertains.

Unless the context indicates otherwise, it is specifically intended that the various features of the disclosure described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination. The term consisting of intends the recited elements and any additional elements that do not materially change of the function of the recited element or elements.

Unless explicitly indicated otherwise, all specified embodiments, features, and terms intend to include both the recited embodiment, feature, or term and biological equivalents thereof.

### Definitions

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compounds, compositions and methods include the recited elements, but not exclude others. “Consisting essentially of when used to define compounds, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like.” Consisting of shall mean excluding more than trace elements of other ingredients. Embodiments defined by each of these transition terms are within the scope of this technology.

All numerical designations, e.g., pH, temperature, time, concentration, and molecular weight, including ranges, are approximations which are varied (+) or (−) by increments of 1, 5, or 10%. It is to be understood, although not always explicitly stated that all numerical designations are preceded by the term “about.” It also is to be understood, although not always explicitly stated, that the reagents described herein are merely exemplary and that equivalents of such are known in the art.

Also as used herein, “and/or” refers to and encompasses any and all possible combinations of one or more of the associated listed items, as well as the lack of combinations when interpreted in the alternative (“or”).

As used herein, the terms “nucleic acid sequence,” “nucleotide sequence,” “polynucleotide,” “polynucleotide sequence” and “oligonucleotide” are used interchangeably to refer to a polymeric form of nucleotides of any length, including but not limited to, deoxyribonucleotides, ribonucleotides, analogs of each thereof, or combinations thereof. Polynucleotides can have any three-dimensional structure and may perform any function, known or unknown. “RNA” is a polynucleotide named ribonucleic acid while “DNA” stands for a polynucleotide named deoxyribonucleic acid. Thus, this term includes, but is not limited to, single-, double-, or multi-stranded DNA or RNA, genomic DNA, complementary DNA (cDNA), DNA-RNA hybrids, synthetic forms, mixed polymers, both sense and antisense strands, or a polymer comprising purine and pyrimidine bases or other natural, chemically or biochemically modified, non-natural, or derivatized nucleotide bases. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. In certain embodiments, the polynucleotide comprises and/or encodes a messenger RNA (mRNA), a short hairpin RNA, and/or small hairpin RNA. In one embodiment, the polynucleotide is or encodes an mRNA. In certain embodiments, the polynucleotide is a single-strand (ss) DNA, such as an engineered ss DNA or an ss cDNA synthesized from a single-stranded RNA. In certain embodiments, the polynucleotide is a DNA/RNA hybrid, such as an engineered ss DNA or an ss cDNA synthesized from a single-stranded RNA and forming a hybrid with the RNA. In certain embodiments, the polynucleotide is a single-stranded RNA. A polynucleotide disclosed herein can be delivered to a cell or tissue or subject using a vector as described herein or other transduction methods known to those of skill in the art.

A polynucleotide can comprise modified nucleotides, such as methylated nucleotides and nucleotide analogs. The modification can be chemical or biochemical and may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component.

As used herein, when referring to the length of a polynucleotide, the unit “nucleotides” i.e., “nt” is used. In the embodiments of a single-strand polynucleotide, the length of the polynucleotide is presented herein as the total number of nucleotide residues that the polynucleotide comprises. In the embodiments of a double-strand or multi-strand polynucleotide, the length of the polynucleotide is presented as the number of the total number of nucleotide residues that the longest strand of the polynucleotide comprises.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The term “isolated” or “recombinant” or “engineered” as used herein with respect to a polynucleotide, such as DNA or RNA, refers to molecules separated from other DNAs or RNAs, respectively that are present in the natural source of the macromolecule as well as polypeptides. The term “isolated or engineered polynucleotide” is meant to include polynucleotide fragments which are not naturally occurring as fragments and would not be found in the natural state. The term “isolated” is also used herein to refer to polynucleotides, polypeptides and proteins that are isolated from other cellular proteins and is meant to encompass both purified and recombinant polynucleotides, polypeptides and proteins. In other embodiments, the term “isolated” means separated from constituents, cellular and otherwise, in which the cell, tissue, polynucleotide, peptide, polypeptide, protein, antibody or fragment(s) thereof, which are normally associated in nature. For example, an isolated cell is a cell that is separated from tissue or cells of dissimilar phenotype or genotype. An isolated polynucleotide is separated from the 3′ and 5′ contiguous nucleotides with which it is normally associated in its native or natural environment, e.g., on the chromosome. As is apparent to those of skill in the art, a non-naturally occurring polynucleotide, peptide, polypeptide, protein, antibody or fragment(s) thereof, does not require “isolation” to distinguish it from its naturally occurring counterpart.

The term ‘isolating” intends the process of separating a composition or component from others in close proximity or contingent therewith. Cells can be isolated manually (e.g., by hand using a pipette or other tool), enzymatically by the use of chemical agents or digitally by the use of digital techniques based on cell or rosette morphology. See, e.g., cellavision.com/en/introducing-digital-cell-morphology-by-cellavision, accessed on May 22, 2018. Polynucleotides (such as a single-stranded RNA, a single-stranded DNA, or a DNA/RNA hybrid) can be isolated, for example, via lysing a cell comprising the polynucleotides, precipitating other cell debris, precipitating all polynucleotides in the cell, and optionally separating the desired polynucleotide (such as via gel separation).

As used herein, the terms “engineered” “synthetic” “recombinant” and “non-naturally occurring” are interchangeable and indicate intentional human manipulation, for example, a modification from its naturally occurring form, and/or a sequence optimization.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. In certain embodiments, default parameters are used for alignment. A non-limiting exemplary alignment program is BLAST, using default parameters. In particular, exemplary programs include BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. Sequence identity and percent identity were determined by incorporating them into clustalW (available at the web address: align.genome.jp, last accessed on Mar. 7, 2011).

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position.

A degree of homology between sequences is a function of the number of matching or homologous positions shared by the sequences. An “unrelated” or “non-homologous” sequence shares less than 40% identity, or alternatively less than 25% identity, with one of the sequences of the present disclosure. As used herein, a sequence (such as a polynucleotide) “homologous” to another sequence refers to the two sequences have shared ancestry and/or share certain identity, such as at least about 50% identity, or alternatively at least about 60% identity, or alternatively at least about 70% identity, or alternatively at least about 80% identity, or alternatively at least about 90% identity, or alternatively at least about 95% identity, or alternatively at least about 96% identity, or alternatively at least about 97% identity, or alternatively at least about 98% identity, or alternatively at least about 99% identity, or alternatively up to 100% identity, optionally over either of the compared sequences (such as the one with the shorter length or the one with the longer length). For example, a sequence homologous to a SOI may share at least about 50% identity, or alternatively at least about 60% identity, or alternatively at least about 70% identity, or alternatively at least about 80% identity, or alternatively at least about 90% identity, or alternatively at least about 95% identity, or alternatively at least about 96% identity, or alternatively at least about 97% identity, or alternatively at least about 98% identity, or alternatively at least about 99% identity, or alternatively up to 100% identity to a segment or the full length of the SOT. As used herein, a segment of a sequence refers to a continuous fragment of the sequence. In a further embodiment, the SOI segment that is used to determine its homology or identity of another sequence is about 1 nt to about 99 nt long. In a further embodiment, the SOI segment is about 10 nt, or alternatively about 20 nt, or alternatively about 30 nt, or alternatively about 40 nt, or alternatively about 50 nt, or alternatively about 60 nt, or alternatively about 70 nt, or alternatively about 80 nt, or alternatively about 90 nt, or alternatively about 100 nt long.

Further, the phrase “terminal redundancy” as used herein refers to a polynucleotide having a segment of its 5′ end homologous to (optionally identical to) a segment of its 3′ end. In one embodiment, the 5′ end of a polynucleotide refers to a segment of the polynucleotide comprising, or alternatively consisting essentially of, or yet further consisting of the first nucleotide residue to the 100^{th} (or alternatively 150^{th}, or alternatively 200^{th}, or alternatively 250^{th}, or alternatively 300^{th}, or alternatively 350^{th}, or alternatively 400^{th}, or alternatively 450^{th}, or alternatively 500^{th}) nucleotide, counting from the polynucleotide end having the terminal phosphate group. In a further embodiment, the 3′ end of a polynucleotide refers to a segment of the polynucleotide comprising, or alternatively consisting essentially of, or yet further consisting of the first nucleotide residue to the 100^{th} (or alternatively 150^{th}, or alternatively 200^{th}, or alternatively 250^{th}, or alternatively 300^{th}, or alternatively 350^{th}, or alternatively 400^{th}, or alternatively 450^{th}, or alternatively 500^{th}) nucleotide, counting from the polynucleotide end having the terminal hydroxyl (OH) group.

As used herein, a sequence (such as a polynucleotide) comprising a component at “the 5′ side or the 3′ side” of another component describes a relative location between the two components in the sequence, i.e. the former component, compared to the latter component, is closer to the terminal phosphate group or the terminal hydroxyl (OH) group, respectively. In one embodiment, the two components do not overlap with each other. In a further embodiment, the first component is adjacent or immediately adjacent to the second component. In another embodiment, the first component is not adjacent to the second component, i.e., the two components are separated via a fragment of the polynucleotide (i.e., an intervening sequence).

As used herein, the term “5′ to 3′” indicates an order of multiple components in a polynucleotide, i.e., from the terminal phosphate end to the terminal hydroxyl (OH) end. Any two of the components can be adjacent to each other, immediately adjacent to each other, or not adjacent to (i.e., having an intervening sequence between) each other. In some embodiment, additional fragment(s) of the polynucleotide may exist in one and/or both of the polynucleotide terminus and/or between any two components.

As used herein, being “adjacent” herein means being within 1 to 8 nucleotides of the site of reference, including being “immediately adjacent,” which means that there is no intervening nucleotides between the immediately adjacent nucleotide sequences and the immediately adjacent nucleotide sequences are within one nucleotide of each other.

“Homology” or “identity” or “similarity” can also refer to two nucleic acid molecules that hybridize under stringent conditions.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. In general, a low stringency hybridization reaction is carried out at about 40° C. in 10×SSC or a solution of equivalent ionic strength/temperature. A moderate stringency hybridization is typically performed at about 50° C. in 6×SSC, and a high stringency hybridization reaction is generally performed at about 60° C. in 1×SSC. Additional examples of stringent hybridization conditions include: low stringency of incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed. Hybridization reactions can also be performed under “physiological conditions” which is well known to one of skill in the art. A non-limiting example of a physiological condition is the temperature, ionic strength, pH and concentration of Mg²⁺ normally found in a cell.

When hybridization occurs in an antiparallel configuration between two single stranded polynucleotides, the reaction is called “annealing” and those polynucleotides are described as “complementary”. A double stranded polynucleotide can be “complementary” or “homologous” to another polynucleotide if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base pairing rules.

In certain embodiments relating to a protein-coding sequence, the term “sense strand” refers to a polynucleotide that carries the translatable code in the 5′ to 3′ direction, and which is complementary to the antisense strand of the polynucleotide, or template strand, which does not carry the translatable code in the 5′ to 3′ direction. In some embodiments relating to a polynucleotide transcribing to an RNA but not encoding a protein, the term “sense strand” refers to a polynucleotide that carries the transcribable code in the 5′ to 3′ direction, and which is complementary to the antisense strand of the polynucleotide, which does not carry the transcribable code in the 5′ to 3′ direction.

In some embodiments, a complementary sequence refers to polynucleotide sequence of bases that can form a double-stranded structure by matching base pairs, for example a reverse complement of the reference sequence. In a further embodiment, the complementary sequence is at least about 90% identical, or alternatively at least about 91% identical, or alternatively at least about 92% identical, or alternatively at least about 93% identical, or alternatively at least about 94% identical, or alternatively at least about 95% identical, or alternatively at least about 96% identical, or alternatively at least about 97% identical, or alternatively at least about 98% identical, or alternatively at least about 99% identical, to the reverse complement of the reference sequence, with proviso that the complementary sequence still hybridizes to the reference sequence.

In some embodiments, a polynucleotide corresponding to an RNA sequence refers to a polynucleotide comprising, or alternatively consisting essentially of, or yet further consisting of the RNA sequence and/or a polynucleotide having at least one U residue of the RNA sequence replaced with a T residue and keeping the remaining residues the same. In one embodiment, a polynucleotide corresponding to the RNA sequence comprises or consists essentially of, or yet further consists of all U residues of the RNA replaced with T residues while keeping the rest residues of the RNA the same.

In some embodiments, an RNA sequence of a gene refers to the RNA polynucleotide encoded by the gene.

As used herein, a regulatory sequence that controls the transcription to the RNA, which is also known as transcriptional regulatory element(s), refers to a polynucleotide sequence that activates transcription alone or in combination with one or more other nucleic acid sequences. A transcriptional regulatory element can, for example, comprise one or more promoters, one or more response elements, one or more negative regulatory elements, one or more enhancers, one or more of U3 that is functional as a promoter, one or more introns, one or more TATA boxes, one or more insulators, one or more silencers, one or more a Woodchuck Hepatitis Virus (WHP) Posttranscriptional Regulatory Elements (WPREs), a 5′ cap, a pA signal and/or a polyadenylation signal sequence encoding a pA signal.

A “promoter” is a control sequence that is a region of a polynucleotide sequence at which initiation and rate of transcription are controlled. It may contain genetic elements at which regulatory proteins and molecules may bind such as RNA polymerase and other transcription factors. In some embodiments, the term “promoter” as used herein refers to any sequence that regulates the expression of a coding sequence, such as a gene. Promoters may be constitutive, inducible, repressible, or tissue-specific. A constitutive promoter refers to one that is always active and/or constantly directs transcription of a gene above a basal level of transcription. In some embodiments, the promoter is a strong, constitutive promoter such as that of cytomegalovirus, simian virus 40, Rous Sarcoma Virus, or the ubiquitin C promoter. An inducible promoter is one which is capable of being induced by a molecule or a factor added to the cell or expressed in the cell. An inducible promoter may still produce a basal level of transcription in the absence of induction, but induction typically leads to significantly more production of the protein. In one embodiment, a promoter that controls target cell specific expression and may be regulatable by transacting molecules.

Examples of promoters include but are not limited to those for RNA Polymerase II and III. RNA Polymerase II or “pol II” promoter catalyzes the transcription of DNA to synthesize precursors of mRNA, and most shRNA and microRNA. Examples of pol II promoters are known in the art and include without limitation, the phosphoglycerate kinase (“PGK”) promoter; EF1-alpha; CMV (minimal cytomegalovirus promoter); MNDU3; and LTRs from retroviral and lentiviral vectors. A RNA Polymerase III or “pol III” promoter is a polynucleotide found in eukaryotic cells that transcribes DNA to synthesize ribosomal 5S rRNA, tRNA and other small RNAs. An examples of pol III promoters include without limitation a U6 promoter.

Non-limiting examples of promoters include cytomegalovirus (CMV), simian virus 40 (SV40), phosphoglycerate kinase 1 (PGK1), ubiquitin C (Ubc), human beta actin, CAG, TRE, UAS, Ac5, polyhedron, CaMKIIa, GAL1, GAL10, TEF1, GDS, ADH1, CaMV35S, Ubi, H1, U6, SSFV, MNDU3, and EF1-α (alternatively named Ef1α). Promoters can also be tissue specific. A tissue specific promoter allows for the production of a protein in a certain population of cells that have the appropriate transcriptional factors to activate the promoter. Numerous promoters are commercially available and widely known in the art; an exemplary sequence can be found at Entrez Gene ID 1915. In one embodiment, the promoter is selected from the group of a cytomegalovirus immediate-early promoter (CMV), a simian virus 40 early promoter (SV40), or a Rous sarcoma virus LTR promoter (RSV).

The target cell specific regulatory elements and promoters include, but are not limited to, one or more elements of the group consisting of Whey Acidic Protein (WAP), Mouse Mammary Tumour Virus (MMTV), β-lactoglobulin and casein specific regulatory elements and promoters, which may be used to target human mammary tumours, pancreas specific regulatory elements and promoters including carbonic anhydrase II and β-glucokinase regulatory elements and promoters, lymphocyte specific regulatory elements and promoters including human immunodeficiency virus (HIV), immunoglobulin and MMTV lymphocytic specific regulatory elements and promoters and MMTV specific regulatory elements and promoters such as MMTVP2 conferring responsiveness to glucocorticoid hormones or directing expression to the mammary gland, T-cell specific regulatory elements and promoters such as T-cell receptor gene and CD4 receptor promoter, B-cell specific regulatory elements and promoters such as immunoglobulin promoter or mb 1. The regulatory elements and promoters regulate preferably the expression of at least one of the coding sequences of a retroviral vector as described herein.

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

As used herein, the term “enhancer”, denotes sequence elements that augment, improve or ameliorate transcription of a nucleic acid sequence irrespective of its location and orientation in relation to the nucleic acid sequence to be expressed. An enhancer may enhance transcription from a single promoter or simultaneously from more than one promoter. As long as this functionality of improving transcription is retained or substantially retained (e.g., at least 70%, at least 80%, at least 90% or at least 95% of wild-type activity, that is, activity of a full-length sequence), any truncated, mutated or otherwise modified variants of a wild-type enhancer sequence are also within the above definition.

As used herein, the term “Woodchuck hepatitis virus post-transcriptional regulatory element” abbreviated as WPRE refers to a sequence that stimulates the expression of a polynucleotide via increased nuclear export.

“Under transcriptional control” is a term well understood in the art and indicates that transcription of a polynucleotide sequence, usually a DNA sequence, depends on its being operatively linked to an element which contributes to the initiation of, or promotes, transcription. “Operatively linked” intends the polynucleotides are arranged in a manner that allows them to function in a cell.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, the term “polyprotein” refers to a large precursor polypeptide that requires proteolytic cleavage into individual (and optionally more than one) smaller polypeptide chains.

Examples of such polyproteins include but are not limited to the precursor polypeptide encoded by the gag gene, the precursor polypeptide encoded by the env gene, and the precursor polypeptide encoded by the pol gene. The resulted smaller proteins are then referred to herein as processed polyproteins.

In some embodiments, the term “processed” when describing a polyprotein indicates one or more peptide bond(s) between amino acids in the precursor polyprotein is/are broken, resulting in smaller polypeptides/proteins.

The phrase “a protein encoded by a gag gene” may refer to one or more of the following: the precursor polyprotein encoded by a gag gene or any one of its processed polyproteins. As used herein, the term “group-specific antigen polyprotein”, also known as gag protein, refers to a polyprotein encoded by the gag gene. Further, the term “processed group-specific antigen polyprotein” refers to the proteins produced by proteolytic cleaving the group-specific antigen polyprotein. Non-limiting examples of the processed group-specific antigen polyproteins include matrix protein (MA), capsid (CA), nucleocapsid (NC), HIV p6, HIV sp1 and HIV sp2.

As used herein, the term “a protein encoded by a pol gene” refers to one or more of the following: the precursor polyprotein encoded by a pol gene or any one of its processed polyproteins. The precursor polyprotein encoded by a pol gene gives rise to one or more of the following processed polyproteins: a protease (PR), a reverse transcriptase (RT), and an integrase (IN). In some embodiments, the RT is the form of the enzyme biologically active with both polymerase and RNase H activity. In a further embodiment, the RT comprises an RNase H domain. PR, RT and IN as used herein may be a wild type or a mutant. See, for example, Blain S W, Goff S P. Differential effects of Moloney murine leukemia virus reverse transcriptase mutations on RNase H activity in Mg2+ and Mn2+. J Biol Chem. 1996; 271(3):1448-1454.

As used herein, the term “a protein encoded by an env gene” which is also referred to herein as an “envelope protein”, may refer to one or more of the following: the precursor polyprotein encoded by an env gene or any one of its processed polyproteins. The precursor polyprotein encoded by an env gene gives rise to one or more of the following processed polyproteins: a surface envelope protein (SU) and a transmembrane envelope protein (TM). The SU and TM form the viral spike protein. SU is responsible for the receptor-binding function of the virus, thus determining the tropism of the virus/viral vector. Based on the cell to be transduced by the viral vector, suitable SU may be chosen by one of skill in the art. In some embodiments, the precursor polyprotein is selected from: glycoprotein 160 (gp160, such as an HIV gp160), glycoprotein 70 (gp70, such as a Mouse Mammary Tumor Virus (MMTV) gp70), and Pr95 (such as an Avian Sarcoma and Leukosis Virus (ASLV) Pr95). In some embodiments, the SU is selected from the following: glycoprotein 120 (gp120, such as an HIV gp120), glycoprotein 52 (gp52, such as MMTV gp52), and glycoprotein 85 (gp85, such as ASLV gp85). In some embodiments, the TM is selected from the following: glycoprotein 41 (gp41, such as an HIV gp41), glycoprotein 36 (gp36, such as MMTV gp36), and glycoprotein 37 (gp37, such as ASLV gp37). Envelop protein is primarily responsible for binding the cellular receptor and for effecting the fusion process, with these functions mediated by protein domains localized to the exterior of the virus. Thus, such envelope proteins may be amphotropic (i.e., recognizing receptors in a broad range of mammalian (host) cell types) and/or ecotropic (i.e., recognizing receptors in tissue culture cells derived from the host species) and/or xenotropic (i.e., recognizing receptors in the cells of a species foreign to the normal host species, a species different from that which normally hosts it).

Retroviruses carry their genetic information in the form of RNA; however, once the virus infects a cell, the RNA is reverse transcribed into the DNA form which integrates into the genomic DNA of the infected cell. The integrated DNA form is called a provirus. In one embodiment, the retrovirus is a virus in the Orthoretrovirinae subfamily or the Spumaretrovirinae subfamily. In a further embodiment, the Orthoretrovirinae subfamily comprises or consists essentially of, or yet further consists of Alpharetrovirus, Betaretrovirus, Deltaretrovirus, Epsilonretrovirus, Gammaretrovirus, or Lentivirus. In yet a further embodiment, the Spumaretrovirinae subfamily comprises or consists essentially of, or yet further consists of Bovispumavirus, Equispumavirus, Felispumavirus, Prosimiispumavirus, or Simiispumavirus. Non-limiting examples of Alpharetrovirus are: Avian carcinoma Mill Hill virus 2, Avian leukosis virus (ALV), Avian myeloblastosis virus, Avian myelocytomatosis virus 29, Avian sarcoma leukosis virus (ASLV), Avian sarcoma virus CT10, Fujinami sarcoma virus, Rous sarcoma virus, UR2 sarcoma virus, or Y73 sarcoma virus. Non-limiting examples of the Betaretrovirus are Langur virus, Mason-Pfizer monkey virus (MPMV), Mouse mammary tumor virus (MMTV), Squirrel monkey retrovirus, or Jaagsiekte sheep retrovirus. Non-limiting examples of Deltaretrovirus are Human T-lymphotropic virus (also named Human T-cell Leukaemia Virus, optionally selected from HTLV-1, HTLV-2, HTLV-3, HTLV-4), adult T-cell leukemia virus (ATLV), Simian-T-lymphotropic virus (types 1-4), Primate T-lymphotropic virus 1, Primate T-lymphotropic virus 2, Primate T-lymphotropic virus 3, or Bovine leukemia virus (BLV). Non-limiting examples of Epsilonretrovirus are Walleye dermal sarcoma virus, Walleye epidermal hyperplasia virus 1, or Walleye epidermal hyperplasia virus 2; wherein the Gammaretrovirus is selected from Chick syncytial virus, Murine Sarcoma Virus (MSV), Finkel-Biskis-Jinkins murine sarcoma virus, Gardner-Arnstein feline, sarcoma virus, Gibbon ape leukemia virus, Guinea pig type-C oncovirus, Hardy-Zuckerman, feline sarcoma virus, Harvey murine sarcoma virus, Kirsten murine sarcoma virus, Moloney murine sarcoma virus, Porcine type-C oncovirus, Reticuloendotheliosis virus, Snyder-Theilen feline sarcoma virus, Trager duck spleen necrosis virus, Viper retrovirus, Woolly monkey sarcoma virus, Murine leukemia virus (MLV), Abelson murine leukemia virus, Friend virus, Feline leukemia virus (FELV), Koala retrovirus (KoRV), or Xenotropic murine leukemia virus-related virus. Non-limiting examples of Lentivirus are human immunodeficiency virus (HIV), human immunodeficiency virus 1, human immunodeficiency virus 2, Simian immunodeficiency virus (SIV), Feline immunodeficiency virus (FIV), Puma lentivirus (PLV), Equine infectious anemia virus (EIAV), Bovine immunodeficiency virus (BIV), Caprine arthritis encephalitis virus, Jembrana disease virus, or Visna-maedi virus. Non-limiting examples of a virus in the Spumaretrovirinae subfamily are Simian foamy virus or Human foamy virus.

As used herein, a retrovirus may refer to any one retrovirus, for example, as disclosed herein, such as a gammaretorvirus or a lentivirus. Therefore, the term “retrovirus” or a grammatical variation thereof may be replaced by any one of the following terms or their grammatical variation: “gammaretrovirus”, “lentivirus”, or any other retroviral subfamily, genera, or species as disclosed herein.

As used herein, lentiviral vector refers to a viral particle capable of introducing exogenous nucleic acid into a cell through a viral or viral-like entry mechanism. A “lentiviral vector” is a type of retroviral vector wellknown in the art that has certain advantages in transducing non-dividing cells as compared to other retroviral vectors. See, Trono D. (2002) Lentiviral vectors, New York: Spring-Verlag Berlin Heidelberg.

In some embodiments, the term retrovirus may refer to a naturally occurring retrovirus, for example, while describing the original source of a retroviral component. In some other embodiments, the term retrovirus may refer to a retroviral vector or particle with intentional human manipulation. Such retrovirus, retroviral vector, or retroviral particle is also termed as a retrovirus-like vector herein. Genomes of the Retroviridae consist of the following components at minimum, in order 5′ to 3′: R-U5-PBS-Psi-Gag-Pol-Env-PPT-U3-R, with more complex viruses encoding additional accessory genes that assist in the viral replicative cycle. The genes gag, pol and env encode viral structural proteins, as well as enzymes necessary to complete the viral life cycle. For example, Group-specific antigen (gag) proteins are major components of the viral capsid, which are about 2000-4000 copies per virion. Gag possesses two nucleic acid binding domains, including matrix (MA) and nucleocapsid (NC). Specifically recognizing, binding, and packaging the retroviral genomic RNA into assembling virions is one of the important functions of Gag protein. Gag interactions with cellular RNAs also regulate aspects of assembly. The expression of gag alone gives rise to assembly of immature virus-like particles that bud from the plasma membrane. Protease (pro) is expressed differently in different viruses. It functions in proteolytic cleavages during virion maturation to make mature gag and pol proteins. Pol proteins are responsible for synthesis of viral DNA and integration into host DNA after infection. Env proteins play a role in association and entry of virions into the host cell. The ability of the retrovirus to bind to its target host cell using specific cell-surface receptors is given by the surface component (SU) of the Env protein, while the ability of the retrovirus to enter the cell via membrane fusion is imparted by the membrane-anchored trans-membrane component (TM). Thus, it is the Env protein that enables the retrovirus to be infectious. Several protein species are associated with the RNA in the retrovirus virion. Nucleocapsid (NC) protein is the most abundant protein, which coats the RNA; while other proteins, present in much smaller amounts and have enzyme activities. Some enzyme activities that are present in the retrovirus virion includes RNA-dependent DNA polymerase (reverse transcriptase; RT), DNA-dependent DNA polymerase, Ribonuclease H (RNase H) Integrase and Protease. The retroviral RNases H encoded by all retroviruses, including HIV have been demonstrated to show three different modes of cleavage: internal, DNA 3′ end-directed, and RNA 5′ end-directed. All three modes of cleavage constitute roles in reverse transcription. Therefore, The RNase H activity is essential in several aspects of reverse transcription.

As used herein, the term “viral pre-integration complex” (PIC) refers to a nucleoprotein complex of viral genetic material and associated viral and host proteins which is capable of inserting a viral genome into a host genome. The PIC forms after uncoating of a viral particle after entry into the host cell. In the case of the human immunodeficiency virus (HIV), the PIC forms after the Reverse Transcription Complex (RTC) has reverse transcribed the viral RNA into DNA. The PIC consists of viral proteins (including Vpr, matrix and integrase), host proteins (including Barrier to autointegration factor 1) and the viral genome. The PIC enters the cellular nucleus through the nuclear pore complex without disrupting the nuclear envelope, thus allowing HIV and related retroviruses to replicate in non-dividing cells. In some embodiments, the term “viral” refers to retroviral. In some embodiments, the viral genetic material comprises or consists essentially of, or yet further consists of an RNA as disclosed herein and/or a polynucleotide as disclosed herein.

In a retrovirus, the RNA genome comprises terminal noncoding regions, which are important in replication, and internal regions that encode virion proteins for gene expression. The 5′ noncoding regions include R, U5, PBS, and Psi. An R region is a short repeated sequence at each end of the genome used during the reverse transcription to ensure correct end-to-end transfer in the growing chain. U5, on the other hand, is a short unique sequence between R and PBS. PBS (primer binding site) consists of about 18 bases complementary to 3′ end of tRNA primer. The term “encapsidation signal,” “Psi,” “retroviral psi packaging element” and “I′ RNA packaging signal” are used herein interchangeably and refers to a cis-acting RNA element involved in regulating the essential process of packaging the retroviral RNA genome into the viral capsid during replication. In one embodiment, Psi is the RNA target site for packaging by nucleocapsid. In one embodiment, a Psi is identified in the genomes of the retroviruses Human immunodeficiency virus (HIV) and Simian immunodeficiency virus (SIV). The 3′ noncoding regions include PPT (polypurine tract), U3, and R. The PPT is a primer for plus-strand DNA synthesis during reverse transcription. U3 is a sequence between PPT and R, which serves as a signal that the provirus can use in transcription. R is the terminal repeated sequence at 3′ end. Any one or more of these noncoding regions or a functional equivalent thereof may be included or replaced with another sequence in the retrovirus-like vector as disclosed herein.

Lentiviruses, as represented by HIV-1, utilize two polypurine tracts for initiation of plus-strand viral DNA synthesis, PPT and cPPT. As used herein, “central polypurine tract” abbreviated as cPPT refers to a recognition site for provial DNA synthesis, increasing transduction efficiency and transgene expression. Additionally, lentiviruses are characterized by a set of regulatory and accessory genes encoded in the viral genome. In some embodiments (for example, those relating to HIV-1), the DNA genome converted from the RNA genome contains the gag, pol, and env genes that are typical of all retroviruses, and two regulatory (tat and rev) and four accessory (vif, vpr, vpu, and nef) genes. These protein-coding regions are flanked by 5′ and 3′ LTR that are required for reverse transcription, integration, and gene expression steps. Viral regulatory protein, Tat is a trans-activator enhancing the viral RNA synthesis from the 5′ LTR. Tat interacts not with DNA, but with an RNA bulge of a stem-loop structure formed at the 5′ end of nascent transcripts, which is known as the transactivation response region (TAR). Binding of Tat to the TAR then recruits an active transcription elongation complex consisting of cyclin T1 (CycT1), CDK9, and some other factors. Subsequently, CDK9 leads the hyperphosphorylation of the C-terminal domain of RNA polymerase II, in turn resulting in a dramatic stimulation of transcriptional processivity. There is then an increase in the partially spiced and unspliced mRNAs along with a concomitant decrease in the multiply spliced mRNAs, which is caused by the accumulation of Rev protein. Rev is also required for the nuclear export of partially spliced and unspliced mRNAs. These classes of viral RNAs contain a highly structured cis-acting element termed Rev response element (RRE) that is located in the env coding region. Rev bears a leucine-rich nuclear export signal (NES) and, via association with the RRE, mediates nuclear-to-cytoplasmic transport of the partially spliced and unspliced RNAs, resulting in production of Gag, Gag-Pol, Env, and accessory proteins. Accessory proteins (i.e. Vif, Vpr, Vpu, and Nef) are dispensable for viral replication in many in vitro cell culture systems, but these proteins are likely to be required for efficient replication and pathogenicity of HIV-1 in vivo.

As used herein, the term “vesicular stomatitis virus G glycoprotein” abbreviated as VSV-G refers to a broad tropism envelope protein which is a typical type III viral fusion protein. A non-limiting example can be found at Ci Y, Yang Y, Xu C, Shi L. Vesicular stomatitis virus G protein transmembrane region is crucial for the hemi-fusion to full fusion transition. Sci Rep. 2018; 8(1):10669. Published 2018 Jul. 13. doi:10.1038/s41598-018-28868-y.

As used herein, a lipid bilayer, which is also known as phospholipid bilayer, refers to a thin amphipathic membrane made of two layers of lipid molecules. These membranes are flat sheets that form a continuous barrier around all cells. The cell membranes of almost all organisms and many viruses are made of a lipid bilayer, as are the nuclear membrane surrounding the cell nucleus, and other membranes surrounding sub-cellular structures.

As used herein, the term “strand transfer” refers to a process in a retrovirus, involving transferring a growing DNA from one locus of the genomic RNA template in the virus to the other, for example, from the 5′ end of the genome to the 3′ R region of the genome.

The terms “equivalent,” “functional equivalent” and “biological equivalent” are used interchangeably when referring to a particular molecule, biological, or cellular material and intend those having minimal homology while still maintaining desired structure or functionality. Non-limiting examples of equivalent polypeptides, include a polypeptide having at least 60%, or alternatively at least 65%, or alternatively at least 70%, or alternatively at least 75%, or alternatively 80%, or alternatively at least 85%, or alternatively at least 90%, or alternatively at least 95% identity thereto or for polypeptide sequences, or a polypeptide which is encoded by a polynucleotide or its complement that hybridizes under conditions of high stringency to a polynucleotide encoding such polypeptide sequences. Conditions of high stringency are described herein and incorporated herein by reference. Alternatively, an equivalent thereof is a polypeptide encoded by a polynucleotide or a complement thereto, having at least 70%, or alternatively at least 75%, or alternatively 80%, or alternatively at least 85%, or alternatively at least 90%, or alternatively at least 95% identity, or at least 97% sequence identity to the reference polynucleotide, e.g., the wild-type polynucleotide. In certain embodiments, the equivalent performs function(s) of its reference at a comparable level, such as at least about 50%, or alternatively at least about 60%, or alternatively at least about 70%, or alternatively at least about 80%, or alternatively at least about 90%, or alternatively at least about 95%, or alternatively up to 100%, or alternatively more than 100%.

As used herein, the term “functional” may be used to modify any molecule, biological, or cellular material to intend that it accomplishes a particular, specified effect.

As used herein, the term “defective” refers to losing the indicated function for at least 10%, or alternatively at least 20%, or alternatively at least 30%, or alternatively at least 40%, or alternatively at least 50%, or alternatively at least 60%, or alternatively at least 70%, or alternatively at least 80%, or alternatively at least 90%, or alternatively at least 91%, or alternatively at least 92%, or alternatively at least 93%, or alternatively at least 94%, or alternatively at least 95%, or alternatively at least 96%, or alternatively at least 97%, or alternatively at least 98%, or alternatively at least 99%, or alternatively up to 100% of the wild type.

As used herein, the term a polynucleotide variant or a polypeptide variant comprises, or consists essentially of, or yet further consists of an equivalent of the polynucleotide or polypeptide, respectively.

The term “IRES” refers to an internal ribosome entry site of viral, prokaryotic, or eukaryotic origin. In some embodiments, an IRES is an RNA element that allows for translation initiation in a cap-independent manner. Common structural features of IRES elements are described in Gritsenko A., et al. (2017) PLoS Comput Biol 13(9): e1005734, incorporated herein by reference. In some embodiment, the IRES is heterologous to one or more of other polynucleotide components which is from or derived from a retrovirus.

As used herein, the term “signal peptide” or “signal polypeptide” intends an amino acid sequence usually present at the N-terminal end of newly synthesized secretory or membrane polypeptides or proteins. It acts to direct the polypeptide to a specific cellular location, e.g. across a cell membrane, into a cell membrane, or into the nucleus. In some embodiments, the signal peptide is removed following localization. Examples of signal peptides are well known in the art. Non-limiting examples are those described in U.S. Pat. Nos. 8,853,381, 5,958,736, and 8,795,965.

As used herein, the term “heterologous” refers to not sharing the same origin. For example, a bovine growth hormone (BGH) polyadenylation (pA) signal is originally from bovine while a PBS is from or derived from a retrovirus, thus the BGH pA signal is heterologous to the PBS.

As used herein, a component is “derived from” a retrovirus if the component is originally obtained from a retrovirus, and optionally modified thereafter.

The term “encode” as it is applied to polynucleotides refers to a polynucleotide which is said to “encode” a polypeptide or a polynucleotide which is said to, if in its native state or when manipulated by methods well known to those skilled in the art, it can be transcribed and/or translated to produce the mRNA for the polypeptide and/or a fragment thereof. Such polynucleotide is also referred to as a coding sequence for the polypeptide or its gene as used herein. The antisense strand is the complement of such a nucleic acid, and the encoding sequence can be deduced therefrom.

The term “polyadenylation signal” (pA signal), as used herein, refers to an RNA element that mediates the addition of a stretch of polyadenine to the 3′end of the mRNA, while a polyadenylation sequence refers to the RNA element of a pA signal and/or a polynucleotide encoding a pA signal. Suitable polyadenylation signals include a simian virus 40 (SV40) pA signal, a SV40 early pA signal, a SV40 late pA signal, a bovine growth hormone (BGH) pA signal, a thymidine kinase (TK) pA signal, a HSV thymidine kinase pA signal, protamine gene pA signal, EIb pA of adeno virus 5 signal, the pA signal of the human variant of growth hormone and the like. In one embodiment, the pA signal may be a strong one or a weak one. In one embodiment, by “strength” (such as strong or weak) when referring to a polyadenylation signal, it refers to how often it triggers cleavage and addition of the polyA tail. “Weak” polyadenylation signals often are read through and do not trigger cleavage and polyA addition. The natural signals of retroviruses and lentiviruses are known to be weak, that is, they are often read through.

As used herein, the term cloning site refers to a polynucleotide (such as a DNA or a double-strand DNA) which can be recognized and cut, optionally by a restriction enzyme, thereby allowing cloning/introducing a further polynucleotide. In one embodiment, the cloning site comprises or consists essentially of, or yet further consists of one site which can be recognized and cut by a restriction enzyme. In another embodiment, the cloning site comprises or consists essentially of, or yet further consists of more than one sites which can be recognized and cut by an restriction enzyme, thus is referred to herein as a multiple cloning site (MCS). In one embodiment, the MCS comprises or consists essentially of, or yet further consists of about 2, about 3, about 4, about 5, about 6, about 7, about 8, about 9, about 10, about 11, about 12, about 13, about 14, about 15, about 16, about 17, about 18, about 19, about 20, about 25, about 30, or more restriction enzyme cut sites. In a further embodiment, the restriction enzyme cut sites are different from each other. In one embodiment, two of the restriction enzyme may be the same with each other. Non-limiting examples of restriction enzymes and the corresponding cut sites can be found at www.sigmaaldrich.com/technical-documents/articles/biology/restriction-enzymes. html, and www.neb.com/tools-and-resources/selection-charts/alphabetized-list-of-recognition-specificities.

Recombineering refers to recombination-mediated genetic engineering and specifies a precise modification of DNA based on homologous recombination systems, as opposed to the older/more common method of using restriction enzymes and ligases to combine DNA sequences in a specified order. Recombineering is widely used for bacterial genetics, in the generation of target vectors for making a conditional mouse knockout, and for modifying DNA of any source often contained on a bacterial artificial chromosome (BAC), among other applications. Briefly, the recombineering is based on homologous recombination mediated by an enzyme, such as in Escherichia coli mediated by bacteriophage proteins, either RecE/RecT from Rac prophage or Redαβδ from bacteriophage lambda, mediating recombination of linear DNA molecules flanked by homology sequences (referred to herein as a pair of “homology arms”) into target DNA sequences. See, for example, Sharan S K, Thomason L C, Kuznetsov S G, Court D L. Recombineering: a homologous recombination-based method of genetic engineering. Nat Protoc. 2009; 4(2):206-223 Schatz, O., Cromme, F. V., Gruninger-Leitch, F., & Le Grice, S. F. (1989). Point mutations in conserved amino acid residues within the C-terminal domain of HIV-1 reverse transcriptase specifically repress RNase H function. FEBS letters, 257(2), 311-314; Halvas, E. K., Svarovskaia, E. S., & Pathak, V. K. (2000). Development of an in vivo assay to identify structural determinants in murine leukemia virus reverse transcriptase important for fidelity. Journal of virology, 74(1), 312-319; Cherepanov, P., Pluymers, W., Claeys, A., Proost, P., De Clercq, E., & Debyser, Z. (2000). High-level expression of active HIV-1 integrase from a synthetic gene in human cells. FASEB journal: official publication of the Federation of American Societies for Experimental Biology, 14(10), 1389-1399; Wisniewski, M., Palaniappan, C., Fu, Z., Le Grice, S. F., Fay, P., & Bambara, R. A. (1999). Mutations in the primer grip region of HIV reverse transcriptase can increase replication fidelity. The Journal of biological chemistry, 274(40), 28175-28184; Rezende, L. F., Curr, K., Ueno, T., Mitsuya, H., & Prasad, V. R. (1998). The impact of multidideoxynucleoside resistance-conferring mutations in human immunodeficiency virus type 1 reverse transcriptase on polymerase fidelity and error specificity. Journal of virology, 72(4), 2890-2895; and Shah, F. S., Curr, K. A., Hamburgh, M. E., Parniak, M., Mitsuya, H., Arnez, J. G., & Prasad, V. R. (2000). Differential influence of nucleoside analog-resistance mutations K65R and L74V on the overall mutation rate and error specificity of human immunodeficiency virus type 1 reverse transcriptase. The Journal of biological chemistry, 275(35), 27037-27044. In one embodiment, the homology arm is about 1 to about 500 nt long. In a further embodiment, the homology arm is about 10 nt to about 100 nt long, including, but not limited to, about 10 nt long, about 20 nt long, about 30 nt long, about 40 nt long, about 50 nt long, about 60 nt long, about 70 nt long, about 80 nt long, about 90 nt long, about 100 nt long.

As used herein, the term “CRISPR” refers to Clustered Regularly Interspaced Short Palindromic Repeats (CRISPR). CRISPR may also refer to a technique or system of sequence specific genetic manipulation relying on the CRISPR pathway. CRISPR can be used to perform gene editing and/or gene regulation, as well as to simply target proteins to a specific genomic location. Gene editing refers to a type of genetic engineering in which the nucleotide sequence of a target polynucleotide is changed through introduction of deletions, insertions, or base substitutions to the polynucleotide sequence. A CRISPR recombinant expression system can be programmed to cleave a target polynucleotide using a CRISPR endonuclease and a guide RNA or a combination of a crRNA and a tracrRNA. A CRISPR system can be used to cause double-strand or single-strand breaks in a target polynucleotide such as DNA or RNA. A CRISPR system can also be used to recruit proteins or label a target polynucleotide. In some aspects, CRISPR-mediated gene editing utilizes the pathways of nonhomologous end-joining (NHEJ) or homologous recombination to perform the edits. Gene regulation refers to increasing or decreasing the production of specific gene products such as protein or RNA. These applications of CRISPR technology are known and widely practiced in the art. See, e.g., U.S. Pat. No. 8,697,359 and Hsu et al. (2014) Cell 156(6): 1262-1278.

The term “guide polynucleotide” as used herein refers to the guide sequences used to target specific genes for correction employing the CRISPR technique. In some embodiments, the guide polynucleotide is a guide RNA (gRNA). Techniques of designing gRNAs and donor therapeutic polynucleotides (i.e., donor template polynucleotide as used herein) for target specificity are well known in the art. For example, Doench, J., et al. Nature Biotechnology 2014; 32(12):1262-7, Mohr, S. et al. (2016) FEBS Journal 283: 3232-38, and Graham, D., et al. Genome Biol. 2015; 16: 260. Additionally or alternatively, guide polynucleotide comprises or alternatively consists essentially of, or yet further consists of a fusion polynucleotide comprising CRISPR RNA (crRNA) and trans-activating CRIPSPR RNA (tracrRNA); or a polynucleotide comprising CRISPR RNA (crRNA) and trans-activating CRIPSPR RNA (tracrRNA). In some aspects, a gRNA is synthetic (Kelley, M. et al. (2016) J. of Biotechnology 233 (2016) 74-83). As used herein, a biological equivalent of a gRNA includes but is not limited to polynucleotides or targeting molecules that can guide a Cas9 or equivalent thereof to a specific nucleotide sequence such as a specific region of a cell's genome.

Editing in cells can be achieved via an expression system consisting of conventional CRISPR/Cas systems, guide RNAs specific to the target genes in the cells, and an optional donor template sequence. The retroviral particle as described herein provides a suitable expression system. It is further appreciated that a CRISPR editing construct may be useful in binding to an endogenous nucleic acid, knocking out an endogenous nucleic acid, or knocking in a nucleic acid. Accordingly, it is appreciated that a CRISPR system can be designed for to accomplish one or both of these purposes.

The term “Cas protein” refers to a CRISPR-associated, RNA-guided endonuclease such as Streptococcus pyogenes Cas9 (spCas9) and orthologs and biological equivalents thereof. Biological equivalents of Cas9 include but are not limited to Type VI CRISPR systems, such as Cas13a, Cas12a (Cpf1), C2c1, C2c2, and Cas13b, which target RNA rather than DNA. In some embodiment, Cas9 is derived from Streptococcus thermophiles, Legionella pneumophilia, Neisseria lactamica, Neisseria meningitides, Francisella novicida; and Cpf1 (which performs cutting functions analogous to Cas9) is derived from various bacterial species including Acidaminococcus spp. and Francisella novicida U112. A Cas protein refers to an endonuclease that causes breaks or nicks in RNA as well as other variations such as dead Cas9 or dCas9, which lack endonuclease activity. In particular embodiments, the Cas protein is modified to eliminate endonuclease activity (referred to herein as “inactivated Cas protein”). For example, both RuvC and HNH nuclease domains can be rendered inactive by point mutations (e.g., D10A and H840A in SpCas9), resulting in a nuclease dead Cas9 (dCas9) molecule that cannot cleave target DNA. The dCas9 molecule retains the ability to bind to target RNA based on the guide RNA targeting sequence. A Cas protein can also refer to a “split” protein in which the protein is split into two halves (e.g., C-Cas9 and N-Cas9) and fused with two intein moieties. See, e.g., U.S. Pat. No. 9,074,199 B1; Zetsche et al. (2015) Nat Biotechnol. 33(2):139-42; Wright et al. (2015) PNAS 112(10) 2984-89.

Non-limiting examples of Cas proteins include Cas 1, Cas1B, Cas2, Cas3, Cas4, Cas5, Cash, Cas7, Cas8, Cas9 (also known as Csn 1 and Csx12), Cas10, Csy1, Csy2, Csy3, Cse1, Cse2, Cscl, Csc2, Csa5, Csn2, Csm2, Csm3, Csm4, Csm5, Csm6, Cmr1, Cmr3, Cmr4, Cmr5, Cmr6, Csbl, Csb2, Csb3, Csx17, Csx14, Csx10, Csx16, CsaX, Csx3, Csx1, Csx15, Csf1, Csf2, Csf3, Csf4, homologues thereof, or modified versions thereof. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of. Orthologous proteins may but need not be structurally related, or are only partially structurally related. Homologs and orthologs may be identified by homology modelling (see, e.g., Greer, Science vol. 228 (1985) 1055, and Blundell et al. Eur J Biochem vol 172 (1988), 513) or “structural BLAST” (Dey F, Cliff Zhang Q, Petrey D, Honig B. Toward a “structural BLAST”: using structural relationships to infer function. Protein Sci. 2013 April; 22(4):359-66. doi: 11.0.1002/pro.2225.). See also Shmakov et al. (2015) for application in the field of CRISPR-Cas loci. Non-limiting examples of Cas9 orthologs include S. aureus Cas9 (“spCas9”), S. thermophiles Cas9, L. pneumophilia Cas9, N. lactamica Cas9, N. meningitides Cas9, B. longum Cas9, A. muciniphila Cas9, and O. laneus Cas9.

As used herein, the term “donor template polynucleotide” refers to a nucleic acid sequence that serves as a template in the process of homologous recombination (optionally caused by a CRISPR system) and that carries the modification that is to be introduced into the target sequence. By using this donor template polynucleotide as a template, the genetic information, including the modification(s), is copied into the target sequence within the genome of the target cell. For example, the donor template polynucleotide can be identical to the part of the target sequence to be replaced, with the exception of one nucleotide that differs and results in the introduction of a point mutation upon homologous recombination or it can comprise an additional gene previously not present in the target sequence. In one embodiment, the template polynucleotide may be a single-stranded polynucleotide. In one embodiment, the donor template polynucleotide comprises regions that are homologous to the target sequence, or to parts of the target sequence.

“Target sequence” refers to a nucleotide sequence adjacent to a 5'-end of a protospacer adjacent motif (PAM). “Target site” refers to a site of the target sequence including both the target sequence and its complementary sequence, for example, in double stranded nucleotides. The target site described herein may mean a nucleotide sequence hybridizing to a sgRNA spacer region, a complementary nucleotide sequence of the nucleotide sequence hybridizing to a sgRNA spacer region, and/or a nucleotide sequence adjacent to the 5′-end of a PAM. Full complementarity of a sgRNA spacer region with a target site is not necessarily required, provided there is sufficient complementarity to cause hybridization and promote formation of a CRISPR complex. A target sequence or target site may comprise any polynucleotide, such as DNA or RNA polynucleotides. In some embodiments, a target sequence or target site is located in the nucleus or cytoplasm of a cell. In some embodiments, the target sequence or target site may be within an organelle of a eukaryotic cell, for example, mitochondrion or chloroplast.

A “protospacer adjacent motif” (PAM) herein refers to a short nucleotide sequence adjacent to a target sequence (protospacer) that is recognized (targeted) by a sgRNA/Cas endonuclease system described herein. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long. The PAM sequence plays a key role in target recognition by licensing sgRNA base pairing to the protospacer sequence (Szczelkun et al, 2014, Proc. Natl. Acad. Sci. U.S.A 111: 9798-803).

In some embodiments, the polynucleotide delivered to a cell and/or a subject comprises one or more of inhibitory RNAs. As used herein, the term “inhibitory RNA” refers to an RNA molecule capable of RNA interference, a mechanism whereby an inhibitory RNA molecule targets a messenger RNA (mRNA) molecule, resulting in inhibition gene expression and/or translation. RNA interference is also known as post-transcriptional gene silencing. Exemplary inhibitory RNAs include but are not limited to antisense RNAs, microRNAs (miRNA), small interfering RNAs (siRNA), short hairpin RNAs (shRNA), double stranded RNA (dsRNA) and intermediates thereof. Methods of designing, cloning, and expressing inhibitory RNAs are known in the art (e.g. McIntyre et al, BMC Biotechnol 2006; 6:1; Moore et al. Methods Mol Biol. 2010; 629: 141-158) and custom RNAi kits are commercially available (e.g. GeneAssist Custom siRNA Builder, ThermoFisher Scientific, Waltham, Mass.).

In one embodiment, the term “siRNA” stands for small interfering RNA which is a non-coding RNA used to interfere with the translation of proteins by binding to and promoting the degradation of messenger RNA (mRNA). In one embodiment, the siRNA is a double strand RNA. The term siRNA also intends short hairpin RNAs (shRNAs). shRNAs comprise a single strand of RNA that forms a stem-loop structure, where the stem consists of the complementary sense and antisense strands that comprise a double-stranded siRNA, and the loop is a linker of varying size. The stem structure of shRNAs generally is from about 10 to about 30 nucleotides long. Non-limiting examples of a suitable siRNA can be found at thermofisher.com/us/en/home/life-science/rnai/synthetic-rnai-analysis.html, sigmaaldrich.com/life-science/functional-genomics-and-rnai/sirna/mission-predesigned-sirna.html and horizondiscovery.com/en/products/gene-modulation/knockdown-reagents/sirna/sirna, as well as in the Table 1.

As used herein, the term “microRNAs” or “miRNAs” refers to post-transcriptional regulators that typically bind to, for example, complementary sequences in the 3′ untranslated regions (3′ UTRs) of target messenger RNA transcripts (mRNAs), thereby degrading their target mRNAs and/or inhibiting their translation, usually resulting in gene silencing. Typically, miRNAs are short, non-coding ribonucleic acid (RNA) molecules, for example, 21 or 22 nucleotides long. The terms “microRNA” and “miRNA” and “miR” are used interchangeably.

One of skill in the art can use methods such as RNA interference (RNAi), CRISPR, TALEN, ZFN or other methods that target specific sequences to reduce or eliminate expression and/or function of proteins. CRISPR, TALEN, ZFN or other genome editing tools can also be used to increase expression and/or function of genes.

As used herein, “RNAi” (RNA interference) refers to the method of reducing or eliminating gene expression in a cell by targeting specific mRNA sequences for degradation via introduction of short pieces of double stranded RNA (dsRNA) and small interfering RNA (such as siRNA, shRNA or miRNA etc.) (Agrawal, N. et al.; Microbiol Mol Biol Rev. 2003; 67:657-685, Arenz, C. et al.; Naturwissenschaften. 2003; 90:345-359, Hannon G J.; Nature. 2002; 418:244-251).

As used herein, “TALEN” (transcription activator-like effector nucleases) refers to engineered nucleases that comprise a non-specific DNA-cleaving nuclease fused to a TALE DNA-binding domain, which can target DNA sequences and be used for genome editing. Boch (2011) Nature Biotech. 29: 135-6; and Boch et al. (2009) Science 326: 1509-12; Moscou et al. (2009) Science 326: 3501. TALEs are proteins secreted by Xanthomonas bacteria. The DNA binding domain contains a repeated, highly conserved 33-34 amino acid sequence, with the exception of the 12th and 13th amino acids. These two positions are highly variable, showing a strong correlation with specific nucleotide recognition. They can thus be engineered to bind to a desired DNA sequence. To produce a TALEN, a TALE protein is fused to a nuclease (N), which is a wild-type or mutated Fokl endonuclease. Several mutations to Fokl have been made for its use in TALENs; these, for example, improve cleavage specificity or activity. Cermak et al. (2011) Nucl. Acids Res. 39: e82; Miller et al. (2011) Nature Biotech. 29: 143-8; Hockemeyer et al. (2011) Nature Biotech. 29: 731-734; Wood et al. (2011) Science 333: 307; Doyon et al. (2010) Nature Methods 8: 74-79; Szczepek et al. (2007) Nature Biotech. 25: 786-793; and Guo et al. (2010) J. Mol. Bio. 200: 96. The Fokl domain functions as a dimer, requiring two constructs with unique DNA binding domains for sites in the target genome with proper orientation and spacing. Both the number of amino acid residues between the TALE DNA binding domain and the Fokl cleavage domain and the number of bases between the two individual TALEN binding sites appear to be important parameters for achieving high levels of activity. Miller et al. (2011) Nature Biotech. 29: 143-8. TALENs specific to sequences in immune cells can be constructed using any method known in the art, including various schemes using modular components. Zhang et al. (2011) Nature Biotech. 29: 149-53; Geibler et al. (2011) PLoS ONE 6: e19509.

As used herein, “ZFN” (Zinc Finger Nuclease) refers to engineered nucleases that comprise a non-specific DNA-cleaving nuclease fused to a zinc finger DNA binding domain, which can target DNA sequences and be used for genome editing. Like a TALEN, a ZFN comprises a Fokl nuclease domain (or derivative thereof) fused to a DNA-binding domain. In the case of a ZFN, the DNA-binding domain comprises one or more zinc fingers. Carroll et al. (2011) Genetics Society of America 188: 773-782; and Kim et al. (1996) Proc. Natl. Acad. Sci. USA 93: 1156-1160. A zinc finger is a small protein structural motif stabilized by one or more zinc ions. A zinc finger can comprise, for example, Cys2His2, and can recognize an approximately 3-bp sequence. Various zinc fingers of known specificity can be combined to produce multi-finger polypeptides which recognize about 6, 9, 12, 15 or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Like a TALEN, a ZFN must dimerize to cleave DNA. Thus, a pair of ZFNs are required to target non-palindromic DNA sites. The two individual ZFNs must bind opposite strands of the DNA with their nucleases properly spaced apart. Bitinaite et al. (1998) Proc. Natl. Acad. Sci. USA 95: 10570-5. ZFNs specific to sequences in immune cells can be constructed using any method known in the art. See, e.g., Provasi (2011) Nature Med. 18: 807-815; Torikai (2013) Blood 122: 1341-1349; Cathomen et al. (2008) Mol. Ther. 16: 1200-7; Guo et al. (2010) J. Mol. Bioi. 400: 96; U.S. Patent Publication 201110158957; and U.S. Patent Publication 2012/0060230.

The term “antisense oligonucleotide” (ASO) refers to a synthetic single strand of nucleic acid that bind to RNA, thereby altering or reducing the expression of the RNA. The ASO generally is from about 5 to about 70 nucleotides in length. For example, the ASO can be 5-50 nucleotides in length, or alternatively, 8-50 nucleotides in length, or alternatively, 15-40 nucleotides in length, or alternatively, 10-30 nucleotides in length, or alternatively, 8-40 nucleotides in length

An “endonuclease” refers to an enzyme that cleaves the phosphodiester bond within a polynucleotide chain. Endonucleases include restriction endonucleases that cleave DNA at specific sites without damaging the bases. Restriction endonucleases include Type I, Type II, Type III, and Type IV endonucleases, which further include subtypes. In the Type I and Type III systems, both the methylase and restriction activities are contained in a single complex. Type I and Type III restriction endonucleases recognize specific recognition sites, but typically cleave at a variable position from the recognition site, which can be hundreds of base pairs away from the recognition site. In Type II systems the restriction activity is independent of any methylase activity, and cleavage typically occurs at specific sites within or near to the recognition site. Most Type II enzymes cut palindromic sequences, however Type Ha enzymes recognize non-palindromic recognition sites and cleave outside of the recognition site, Type IIb enzymes cut sequences twice with both sites outside of the recognition site, and Type IIs enzymes recognize an asymmetric recognition site and cleave on one side and at a defined distance of about 1-20 nucleotides from the recognition site. Type IV restriction enzymes target methylated DNA. Restriction enzymes are further described and classified, for example in the REBASE database (webpage at rebase.neb.com; Roberts et al., (2003) Nucleic Acids Res 31:418-20), Roberts et al., (2003) Nucleic Acids Res 31:1805-12, and Belfort et al., (2002) in Mobile DNA II, pp. 761-783, Eds. Craigie et al., (ASM Press, Washington, D.C.).

As used herein, the term “detectable marker” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, β-galactosidase, glucose-6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties. Such detection may be accomplished by optical methods such as diffraction, surface plasmon resonance, surface variation, the contact angle change or physical methods such as atomic force spectroscopy, tunnel effect, or radioactive molecules such as ³²P, ³⁵S or ¹²⁶I. In one embodiment, the detectable marker is a protein or polypeptide expressed from a nucleic acid. In another embodiment, the detectable marker is a polynucleotide encoding a protein or polypeptide.

As used herein, the term “selection marker,” “purification marker” or “reporter protein” refer to at least one marker useful for purification or identification. A non-exhaustive list of this marker includes His, lacZ, GST, maltose-binding protein, NusA, BCCP, c-myc, CaM, FLAG, thioredoxin, poly(NANP), V5, Snap, hemmaglutinin (HA) tag, chitin-binding protein, Softag 1, Softag 3, Strep, S-protein, YUC or a fluorescence marker. Suitable direct or indirect fluorescence marker comprise FLAG, a green fluorescent protein (GFP), an enhanced green fluorescent protein (EGFP), a red flouresence protein (RFP), and yellow fluorescent protein (YFP), RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye, hapten. These are commercially available and described in the technical art. In one embodiment, the selection marker is a protein or polypeptide expressed from a nucleic acid. In another embodiment, the selection marker is a polynucleotide encoding a protein or polypeptide.

A polynucleotide disclosed herein can be delivered to a cell or tissue or subject using a gene delivery vehicle. “Gene delivery,” “gene transfer,” “transduction,” and the like as used herein, are terms referring to the introduction of an exogenous polynucleotide (sometimes referred to as a “transgene”) into a cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the cell or integrates into a replicon of the cell such as an extrachromosomal replicon (e.g., a plasmid) or a nuclear or mitochondrial chromosome. A number of vectors are known to be capable of mediating transfer of genes to mammalian cells, as is known in the art and described herein.

As used herein, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

Moreover, certain vectors are capable of directing the expression of genes to which they are operatively linked. Such vectors are referred to herein as “expression vectors.” Common expression vectors of utility in recombinant DNA techniques are often in the form of plasmids. Recombinant expression vectors can comprise a nucleic acid of the disclosure in a form suitable for expression of the nucleic acid in a cell, which means that the recombinant expression vectors include one or more regulatory elements, which may be selected on the basis of the cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous viral expression vectors include retroviruses, replication defective retroviruses, adenoviruses, replication defective adenoviruses, lentiviruses, replication defective lentiviruses, and adeno-associated viruses.

Vectors may be viral or non-viral.

A “viral vector” is defined as a recombinantly produced virus or viral particle that comprises a polynucleotide to be delivered into a cell, either in vivo, ex vivo or in vitro. In some embodiments, viral vectors may be prepared from commercially available vectors. In other embodiments, viral vectors may be produced from baculoviruses, retroviruses, adenoviruses, AAVs, etc. according to techniques known in the art. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. Further details as to modern methods of vectors for use in gene transfer may be found in, for example, Kotterman et al. (2015) Viral Vectors for Gene Therapy: Translational and Clinical Outlook Annual Review of Biomedical Engineering 17. Vectors that contain both a promoter and a cloning site into which a polynucleotide can be operatively linked are well known in the art. Such vectors are capable of transcribing RNA in vitro or in vivo and are commercially available from sources such as Agilent Technologies (Santa Clara, Calif.) and Promega Biotech (Madison, Wis.).

Adenoviruses (Ads) are a relatively well characterized, homogenous group of viruses, including over 50 serotypes. See, e.g., PCT International Application Publication No. WO 95/27071. Ads do not require integration into the host cell genome. Recombinant Ad derived vectors, particularly those that reduce the potential for recombination and generation of wild-type virus, have also been constructed. See, PCT International Application Publication Nos. WO 95/00655 and WO 95/11984, Wild-type AAV has high infectivity and specificity integrating into the host cell's genome. See, Hermonat & Muzyczka (1984) Proc. Natl. Acad. Sci. USA 81:6466-6470 and Lebkowski et al. (1988) Mol. Cell. Biol. 8:3988-3996.

That the vector particle according to the disclosure is “based on” a particular retrovirus means that the vector is derived from that particular retrovirus. The genome of the vector particle comprises components from that retrovirus as a backbone. The vector particle contains essential vector components compatible with the RNA genome, including reverse transcription and integration systems. Usually these will include gag and pol proteins derived from the particular retrovirus. Thus, the majority of the structural components of the vector particle will normally be derived from that retrovirus, although they may have been altered genetically or otherwise so as to provide desired useful properties. However, certain structural components and in particular the env proteins, may originate from a different virus. The vector host range and cell types infected or transduced can be altered by using different env genes in the vector particle production system to give the vector particle a different specificity.

The term “an expression control element” as used herein, intends a polynucleotide that is operatively linked to a target polynucleotide to be transcribed, and facilitates the expression of the target polynucleotide. A promoter is an example of an expression control element.

Exemplary non-viral vectors for delivering nucleic acid include naked DNA (such as plasmids or YAC); DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; the use of ternary complexes comprising a virus and polylysine-DNA; inorganic particles, calcium phosphate particles, silica nanoparticles, gold nanoparticles, nanoparticles, cationic lipids, lipid nano emulsions, solid lipid nanoparticles, peptide based vectors, polymer based vectors, liposomes, or gelatin-based vectors.

“Plasmids” used in genetic engineering are called “plasmid vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene. This is a cheap and easy way of mass-producing a gene or the protein it then codes for.

A “yeast artificial chromosome” or “YAC” refers to a vector used to clone large DNA fragments (larger than 100 kb and up to 3000 kb). It is an artificially constructed chromosome and contains the telomeric, centromeric, and replication origin sequences needed for replication and preservation in yeast cells. Built using an initial circular plasmid, they are linearized by using restriction enzymes, and then DNA ligase can add a sequence or gene of interest within the linear molecule by the use of cohesive ends. Yeast expression vectors, such as YACs, YIps (yeast integrating plasmid), and YEps (yeast episomal plasmid), are extremely useful as one can get eukaryotic protein products with posttranslational modifications as yeasts are themselves eukaryotic cells, however YACs have been found to be more unstable than BACs, producing chimeric effects.

Gene delivery vehicles also include DNA/liposome complexes, micelles and targeted viral protein-DNA complexes. Liposomes that also comprise a targeting antibody or fragment thereof can be used in the methods disclosed herein. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins disclosed herein are other non-limiting techniques.

The term “cell” as used herein may refer to either a prokaryotic or eukaryotic cell, optionally obtained from a subject or a commercially available source.

“Eukaryotic cells” comprise all of the life kingdoms except monera and archaea. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” or “packaging cell” includes a eukaryotic host, including, for example, yeast, higher plant, insect and mammalian cells. Non-limiting examples of eukaryotic cells or hosts include simian, bovine, porcine, murine, rat, avian, reptilian and human, e.g., HEK293 cells, 293T cells, psi-2, psi-Crypt, psi-AM, GP+E-86, PA317, GP+envAM-12, Fly A13, BOSC 23, BING, Fly RD 18, ProPak-X, -A.52 and -A.6. In certain embodiment, the host cell is selected based on the target cell of a retroviral particle produced by the host cell. Without wishing to be bound by the theory, high similarity between cell membranes of the target cell and the host cell facilitates transduction of the retroviral particle into the target cell. For example, if the target cell of a retroviral particle is a human B cell, a host cell producing such retroviral particle may be selected from a human B cell line.

“Prokaryotic cells” that usually lack a nucleus or any other membrane-bound organelles and are divided into two domains, bacteria and archaea. In addition to chromosomal DNA, these cells can also contain genetic information in a circular loop called a plasmid. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

A “target cell” as used herein, shall intend a cell to be delivered with a polynucleotide as disclosed herein or a retroviral particle as disclosed herein.

The term “express” refers to the production of a gene product. A “gene product” or alternatively a “gene expression product” refers to the amino acid (e.g., peptide or polypeptide) generated when a gene is transcribed and translated.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

The term “introduce” or “deliver” refers to the process whereby a foreign (i.e. extrinsic or extracellular) agent (for example, a polynucleotide or a vector) is introduced into a cell. Methods of introducing nucleic acids include but are not limited to transduction, retroviral gene transfer, transfection, electroporation, transformation, viral infection, and other recombinant DNA techniques known in the art. In some embodiments, transduction is done via a vector (e.g., a viral vector or a non-viral vector). In some embodiments, transfection is done via a non-viral vector, for example, a siRNA-lipid complex, a nanoparticle (non-limiting examples of which includes a gold nanoparticle, a lipid nanoparticle and a polymer-based nanoparticle), an antibody conjugate, a chemical carrier, DNA/liposome complex, micelle (e.g., Lipofectamine (Invitrogen)) or any small molecules that improving oligonucleotide delivery. In some embodiments, viral infection (i.e. transduction) is done via infecting the cells with a viral particle. Methods of introducing non-nucleic acid foreign agents (e.g., soluble factors, cytokines, proteins, peptides, enzymes, growth factors, signaling molecules, small molecule inhibitors) include but are not limited to culturing the cells in the presence of the foreign agent, contacting the cells with the agent, contacting the cells with a composition comprising the agent and an excipient, and contacting the cells with vesicles or viral particles comprising the agent.

The term “culturing” refers to the in vitro propagation of cells or organisms on or in media of various kinds. It is understood that the descendants of a cell grown in culture may not be completely identical (i.e., morphologically, genetically, or phenotypically) to the parent cell. By “expanded” is meant any proliferation or division of cells. A “cultured” cell is a cell that has been separated from its native environment and propagated under specific, pre-defined conditions. Such culture may be performed in a bioreactor supporting a biologically active environment (e.g., temperature, O₂% and CO₂%). In one embodiment, the bioreactor is a closed and/or continuous bioreactor. Additionally or alternatively, the bioreactor is a three dimensional bioreactor.

The term “culturing” includes, but is not limited to, growing cells in a culture medium under conditions that favor expansion and proliferation of the cell. The term “culture medium” or “medium” is recognized in the art and refers generally to any substance or preparation used for the cultivation of living cells. The term “medium”, as used in reference to a cell culture, includes the components of the environment surrounding the cells. Media may be solid, liquid, gaseous or a mixture of phases and materials. Media include liquid growth media as well as liquid media that do not sustain cell growth. Media also include gelatinous media such as agar, agarose, gelatin and collagen matrices. Exemplary gaseous media include the gaseous phase to which cells growing on a petri dish or other solid or semisolid support are exposed. The term “medium” also refers to material that is intended for use in a cell culture, even if it has not yet been contacted with cells. In other words, a nutrient rich liquid prepared for culture is a medium. Similarly, a powder mixture that when mixed with water or other liquid becomes suitable for cell culture may be termed a “powdered medium.” “Defined medium” refers to media that are made of chemically defined (usually purified) components. “Defined media” do not contain poorly characterized biological extracts such as yeast extract and beef broth. “Rich medium” includes media that are designed to support growth of most or all viable forms of a particular species. Rich media often include complex biological extracts. A “medium suitable for growth of a high-density culture” is any medium that allows a cell culture to reach an OD600 of 3 or greater when other conditions (such as temperature and oxygen transfer rate) permit such growth. The term “basal medium” refers to a medium which promotes the growth of many types of microorganisms which do not require any special nutrient supplements. Most basal media generally comprise of four basic chemical groups: amino acids, carbohydrates, inorganic salts, and vitamins. A basal medium generally serves as the basis for a more complex medium, to which supplements such as serum, buffers, growth factors, lipids, and the like are added. In one aspect, the growth medium may be a complex medium with the necessary growth factors to support the growth and expansion of the cells of the disclosure while maintaining their self-renewal capability. Examples of basal media include, but are not limited to, Eagles Basal Medium, Minimum Essential Medium, Dulbecco's Modified Eagle's Medium, Medium 199, Nutrient Mixtures Ham's F-10 and Ham's F-12, McCoy's 5A, Dulbecco's MEM/F-I 2, RPMI 1640, and Iscove's Modified Dulbecco's Medium (IMDM).

As used herein the terms “purification”, “purifying”, or “separating” refer to the process of isolating one or more component from a complex mixture, such as a cell lysate or a mixture of polypeptides or polynucleotides. Non-limiting examples of the component include polynucleotides, such as DNA or RNA, or protein or polypeptide, or lipid rafts or plasma membrane, or viral particles, or cell or cellular organelle, or tissue or organ, separated from other polynucleotides, or proteins or polypeptides, or cells or cellular organelles, or tissues or organs, The purification, separation, or isolation need not be complete, i.e., some other components of the complex mixture may remain after the purification process. However, the product of purification should be enriched for the component relative to the complex mixture before purification and a significant portion of the other components initially present within the complex mixture should be removed by the purification process. As used herein, the term “purified” does not require absolute purity; rather, it is intended as a relative term. Thus, for example, a purified polynucleotide, peptide, protein, biological complexes (such as a retroviral particle) or other active compound is one that is isolated in whole or in part from proteins or other contaminants. Generally, substantially purified polynucleotides, peptides, proteins, biological complexes (such as a retroviral particle), or other active compounds for use within the disclosure comprise more than 80% of all macromolecular species present in a preparation prior to admixture or formulation of the peptide, protein, biological complex or other active compound with a pharmaceutical carrier, excipient, buffer, absorption enhancing agent, stabilizer, preservative, adjuvant or other co-ingredient in a complete pharmaceutical formulation for therapeutic administration. More typically, the polynucleotides, peptide, protein, biological complex (such as a retroviral particle) or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

As used herein the term “concentrate” or a grammatical variation thereof refers to the process of increasing concentration or percentage of a component in a composition. Such process may be performed by ultracentrifugation and/or ultrafiltration and/or precipitation. Kits for concentrating a retroviral particle are also available. See, for example, systembio.com/shop/retro-concentin/, www.cellbiolab s.com/sites/default/files/9F3AAFD7-3048-812A-2EB249DB2CF9348B.pdf, and www.cellbiolabs.com/retrovirus-rapid-quantitation-kit.

An agent of the present disclosure can be administered to a subject by any suitable route of administration. It will also be appreciated that the optimal route will vary with the condition and age of the recipient, and the disease being treated. Administration may be effected by any method that enables delivery of the agent to the site of action. These methods include oral routes, intraduodenal routes, parenteral injection (including intravenous, subcutaneous, intramuscular, intravascular or infusion), topical, and rectal administration. Bolus doses can be used, or infusions over a period of 1, 2, 3, 4, 5, 10, 15, 20, 30, 60, 90, 120 or more minutes, or any intermediate time period can also be used, as can infusions lasting 3, 4, 5, 6, 7, 8, 9, 10. 12, 14 16, 20, 24 or more hours or lasting for 1-7 days or more. Infusions can be administered by drip, continuous infusion, infusion pump, metering pump, depot formulation, or any other suitable means. Administration or treatment in “combination” refers to administering two agents such that their pharmacological effects are manifest at the same time. Combination does not require administration at the same time or substantially the same time, although combination can include such administrations.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

As used herein, the term “animal” refers to living multi-cellular vertebrate organisms, a category that includes, for example, mammals and birds. The term “mammal” includes both human and non-human mammals.

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. In some embodiments, a subject is a human. In some embodiments, a subject has or is diagnosed of having or is suspected of having a disease.

A “composition” is intended to mean a combination of an agent as disclosed herein and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The compositions used in accordance with the disclosure, including cells, treatments, therapies, agents, drugs and pharmaceutical formulations can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described herein.

A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. In one embodiment, one will desire to administer an amount of the compound that is effective to achieve a serum level commensurate with the concentrations found to be effective in vivo. These considerations, as well as effective formulations and administration procedures are well known in the art and are described in standard textbooks.

“Therapeutically effective amount” of a drug or an agent refers to an amount of the drug or the agent that is an amount sufficient to obtain a pharmacological response; or alternatively, is an amount of the drug or agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose, and may occur only after administration of a series of doses. Thus, a therapeutically effective amount may be administered in one or more administrations.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, injection, and topical application.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor. In one aspect, treatment excludes prophylaxis.

The terms “disease” “disorder” and “condition” are used interchangeably herein, referring to a disease, a status of being diagnosed with the disease, or a status of being suspect of having the disease.

In some embodiments, the RNA, polynucleotide, vector, retroviral particle, or any other composition as disclosed herein is used to treat a disease. In one embodiment, the disease is a genetic disease, which intends a disease caused in whole or in part by a change in the DNA sequence away from the normal sequence. As defined by the National Human Genome Research Institute (genome.gov), genetic disorders can be caused by a mutation in one gene (monogenic disorder), by mutations in multiple genes (multifactorial inheritance disorder), by a combination of gene mutations and environmental factors, or by damage to chromosomes (changes in the number or structure of entire chromosomes, the structures that carry genes). As used herein, the genetic diseases include inherited or acquired from during a patient's life. Non-limited examples of genetic disorders include sickle cell disease, DMD, LSDs, cystic fibrosis, Tay-Sachs disease, Hemophilia, osteogenesis imperfecta (01), thalassemia, spinal muscular atrophy, severe intrauterine growth restriction, MPS, Fragile X syndrome, or Huntington's disease

In some embodiments, the RNA, polynucleotide, vector, retroviral particle, or any other composition as disclosed herein is used to deliver a potential vaccine and/or adjuvant (for example, in the form of a single-stranded DNA or a DNA/RNA hybrid) to a subject, thereby preventing a subject from having and/or developing a disease (a preventive vaccine) and/or treating a subject having or suspected of having a disease (a therapeutic vaccine). In one embodiment, the disease is an infection. As used herein, an infection refers to the invasion and multiplication of microorganisms such as bacteria, viruses, and parasites that are not normally present within the body. In one embodiment, the disease is an influenza. In another embodiment, the disease is an infection of a coronavirus. In a further embodiment, the coronavirus is Severe acute respiratory syndrome (SARS) associated coronavirus (SARS-CoV). In yet a further embodiment, the coronavirus is SARS-CoV-2.

### Modes for Carrying Out the Disclosure

The present disclosure aims to provide an additional, more efficient means of delivery of RNA-DNA hybrids and/or single-stranded DNAs to a cell (such as a mammalian cell) that is at least as safe as other viral vector-based systems that exist in the art.

In one aspect, the present disclosure relates to novel RV- and LV-like vectors capable of mediating transfer of sequences of interest in forms other than the usual double-stranded DNA, especially RNA-DNA hybrids and single-stranded DNA, that are preferably unflanked on at least one end by viral sequences. Such novel vectors would allow for more efficient transfer of a polynucleotide and therefore a broader exploitation of the biological processes mediated by them, e.g., gene editing, RNA interference, and antisense inhibition, toward modification of mammalian cells.

Extensive research was carried out into the potential applicability of polynucleotides other than the more common forms of DNA and RNA to precision medicine and biotechnology. Numerous groups have demonstrated the role of RNA-DNA hybrids in DNA double-stranded break repair (Ohle, Tesorero, Schermann, Dobrev, Sinning & Fischer, 2016; Keskin, et al., 2014), for example, suggesting potential applicability of these hybrids to the growing field of gene editing. Additionally, others have shown that hybrids may be of considerable utility in the burgeoning area of RNA interference (Afonin et al., 2013; Afonin et al., 2013). Finally, single-stranded DNA oligonucleotides have proven enormously useful in antisense therapy and are known to work well as donor templates for gene editing.

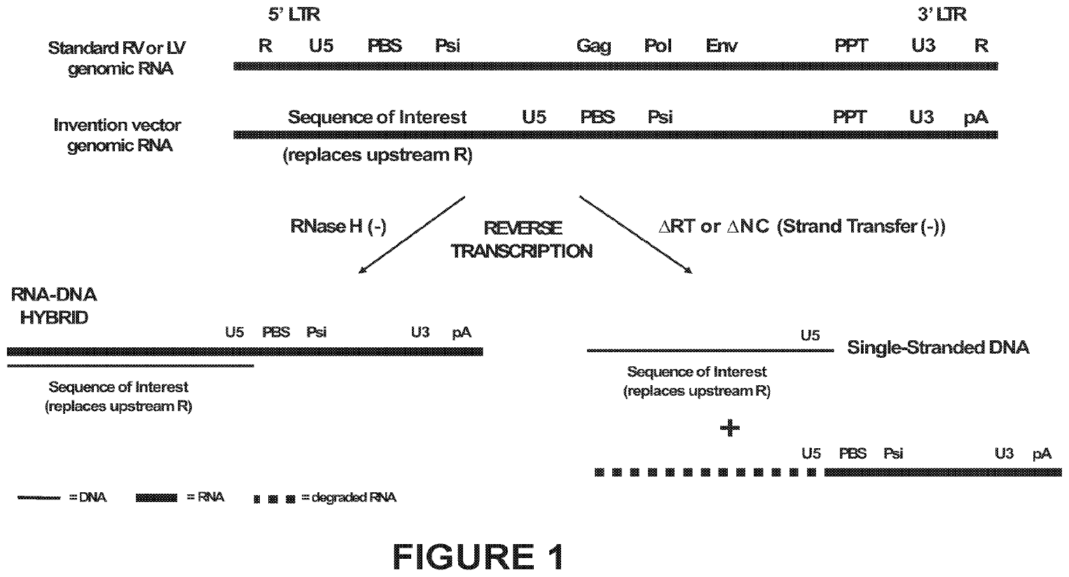

Thus, in a further aspect, provided herein is an RV- or LV-like vector comprising one or more SOIs inserted in sense and/or antisense orientation within the 5′ LTR region and especially lacking any sequence homologous to the SOI within the 3′ LTR region, thus ensuring that the SOI will be at least partly reverse transcribed through the strong stop, but precluding the strong stop jump itself. See FIG. 1. To further reduce the likelihood of the jump occurring, the vector may be packaged with a RT that has been mutated (e.g., L92P and/or F61A in LV and Y598V in RV), rendering it defective in its ability to mediate the strand transfer. Additionally, to produce RNA-DNA hybrids, copackaged RT will have been mutated (e.g., Y586F, D524N, Δ5E, 4C, and/or H7 in RV, or E478Q in LV) to inactivate its RNase H domain.

Whether to clone the SOIs in sense and/or antisense orientation is preferably determined empirically by the user, depending primarily on the presence of transcriptionally active elements in the SOIs that might interfere with the vector life cycle, e.g., polyadenylation sites and/or splice donors, in one or the other orientation. The titer of the vector when the SOI is in either orientation will likely be determinative.

Regarding the safety of the embodiments of this disclosure, specifically concerning the aforementioned possibility of illegitimate integration of the vector reverse transcript into the host cell genome, without wishing to be bound by the theory, SOIs in the form of RNA-DNA hybrids and single-stranded DNA are exceedingly unlikely to spontaneously integrate into the chromosomal DNA. However, an additional safety feature of the disclosed embodiments is the option of copackaging integrases that have been mutated to eliminate their primary activity. Such integrases are common in the art.

To generate the RV- or LV-like particle and the packaged vector, respectively, the principle of a retroviral vector system is used and provided herein. This system consists of two components: the retroviral vector itself in which the genes encoding the viral proteins have been replaced, and a packaging cell line which provides the modified retrovirus with the missing viral proteins. This packaging cell line has been transfected with one or more plasmids carrying the genes enabling the modified retroviral vector to be packaged, but lacks the ability to produce replication competent viruses.

After introduction of the vector into the packaging cell line, the retroviral vector is transcribed into RNA. This transcription is regulated by the normal unselective retroviral promoter contained in the U3 region of the 5′ LTR (or an exogenous promoter,) and optionally initiates immediately downstream of the promoter and terminates downstream of the U3 of the 3′ LTR, as determined by the included pA signal. The RNA which represents the recombinant retroviral genome is packaged by the viral proteins produced by the packaging cell line to form retroviral particles which bud from the cell. These are used to infect the target cell.

After infection of the cell, the recombinant viral RNA is reverse transcribed into DNA (see the FIG. 1). In vectors packaged with either a wildtype RT or RT defective in its ability to mediate strand transfer, the SOI and U5 region should be reverse transcribed to single-stranded DNA. In those vectors packaged with a RT defective in its RNase H activity, the genomic RNA is retained, and the SOI and U5 region are thus in RNA-DNA hybrid form. Regardless of the system used, fidelity of the RT polymerase activity is of vital importance, so embodiments of this disclosure can include either RTs of members of the Retroviridae with naturally high fidelity or RTs of lower fidelity viruses that have been mutated to improve their fidelity.

In one embodiment of this disclosure, the SOI is inserted within the R region of the 5′ LTR, optionally wholly replacing the R region. Hence, transcription of the vector would initiate at the SOI, and the SOI would thereby comprise the 5′ end of the vector RNA. The R region of the 3′ LTR preferably has been wholly or partly replaced by an exogenous polyadenylation sequence, e.g., the SV40 pA signal, the BGH pA signal, the TK pA signal, to properly terminate vector genomic transcripts. Any sequence can be cloned in to replace the 3′ R region, as long as said sequence lacks any homology to the SOI cloned into the 5′LTR, and preferably additionally encodes a polyadenylation sequence.

In a further embodiment, the SOIs comprise, or consist essentially of, or yet further consists of heterologous DNA. The term “heterologous” is used for any combination of DNA sequences that is not normally found intimately associated in nature. As RT naturally produces reverse transcripts of considerable length (many kilobases), it follows that the SOIs can also be produced as RNA-DNA hybrids and single-stranded reverse transcripts of great length, subject only to the packaging capacity of the virion. Hence, the embodiments of this disclosure are intended to accommodate SOIs of a large variety of lengths.

The LTR regions are preferably, but not limited, selected from at least one element of the group consisting of LTR's of (the Retroviridae, e.g.,) Murine Leukaemia Virus (MLV), Mouse Mammary Tumour Virus (MMTV), Murine Sarcoma Virus (MSV), Simian Immunodeficiency Virus (SIV), Human Immunodeficiency Virus (HIV), Human T-cell Leukaemia Virus (HTLV), Feline Immunodeficiency Virus (Hy), Feline Leukaemia Virus (FELV), Bovine Leukaemia Virus (BLV) and Mason-Pfizer-Monkey Virus (MPMV).

In a further embodiment of this disclosure, an RV- and/or LV-like vector system is provided comprising an RV- and/or LV-like vector as described above as a first component and a packaging cell line harboring at least one RV- and/or LV-like and/or recombinant RV- and/or LV-like construct coding for proteins required for said vector to be packaged.

The packaging cell line harbors RV- and/or LV-like or recombinant RV- and/or LV-like constructs coding for those RV- and/or LV-like proteins which are not encoded in said RV- and/or LV-like vector. In some embodiments, the packaging cell line is selected from an element of the group consisting of psi-2, psi-Crypt, psi-AM, GP+E-86, PA317, GP+envAM-12, Fly A13, BOSC 23, BING, Fly RD 18, ProPak-X, -A.52 and -A.6, or of any of these supertransfected with recombinant constructs allowing expression of surface proteins from other enveloped viruses.

After introducing the RV- and/or LV-like vector of the disclosure as described above in a RV- and/or LV-like packaging cell line an RV- and/or LV-like particle is provided comprising the recombinant RV- and/or LV-like genome.

In a further embodiment of this disclosure, a retroviral vector system is provided comprising an RNA and/or a vector as described herein and a packaging cell line harboring at least one retroviral and/or recombinant retroviral construct coding for proteins required for said retroviral vector to be packaged.

The disclosure includes also an RV- and/or LV-like provirus, mRNA of an RV- and/or LV-like provirus according to the disclosure, any RNA resulting from an RV- and/or LV-like vector according to the disclosure and cDNA thereof, as well as cells infected with this disclosure.

This disclosure includes also a retroviral provirus, mRNA of a retroviral provirus according to the disclosure, any RNA resulting from a retroviral vector according to this disclosure and cDNA thereof, as well as host cells infected with a retroviral particle according to the disclosure.

A further embodiment of this disclosure provides a method for removing all remaining viral or undesired sequences from the delivered SOI. An endonuclease that cleaves polynucleotides of one of the relevant forms, i.e., RNA-DNA hybrids and/or single-stranded DNA, is cloned into the body of the vector and expressed using a translation initiation element, e.g., internal ribosomal entry site (Sulej, Tuszynska, Skowronek, Nowotny & Bujnicki, 2012). Expression of the endonuclease therefore removes extraneous sequences from the SOI and thus unencumbers the polynucleotide of the superfluous vector sequences that might otherwise interfere with its activity.

In some embodiments, the RV- and/or LV-like vector, the vector system and the RV and/or LV-like provirus as well as RNA thereof is used for producing a pharmaceutical composition for in vivo and in vitro gene therapy in mammals including humans.

In some embodiments, the vectors or particles as disclosed herein are engineered to be replication defective. In one embodiment, this is accomplished by splitting the essential viral elements onto separate vector-encoding constructs for production purpose. Most commonly, the gag and pol genes are inserted into one expression vector, while env would be cloned into another. A third viral construct encodes the sequence of interest, flanked by all the elements required to be in cis with the SOI, e.g., LTRs, to enable its vector-mediated transfer. In some embodiments and aspects relating to production, all three constructs are introduced/transfected/transduced into cultured cells permissive for viral production. After sufficient time had passed to allow these “producer” cells to generate adequate amounts of vector, such as 48-72 hours, the culture supernatant is harvested and the vector concentrated and purified, depending on application.

In some aspects and/or embodiments, the present disclosure relates to a retroviral vector/particle which is especially applicable as a safer gene transfer vehicle for targeted gene therapy. The vector enables the expression of, e.g., toxic genes, the expression of genes with a specific function that is incompatible with virus vector production and the expression of genes causing vector rearrangements necessary for specific biochemical operations.

In some embodiments, the SOI of an RNA as disclosed herein lacks a promoter and/or a coding sequence encoding a protein.

Additionally, the retroviral vector may comprise one or more elements regulating expression of the coding sequences.

A further embodiment of the disclosure provides a method for introducing homologous and/or heterologous nucleotide sequences into target cells (as e.g. CRFK, NIH/3T3, T-47D, RAT2, MV 1 LU (NBL-7), T-24, PG13, PA317, A20, HT-1080, PANC-1, MIA PA CA-2, HEP G2, 293, MCF-7, CV-1, COS 1, COS 7, FLY2A1) comprising, or consisting essentially of, or yet consisting of infecting a target cell population in vivo and in vitro with recombinant retroviral particles produced by the packaging cell line.

The retroviral vector, the retroviral vector system and the retroviral provirus as well as RNA thereof is used for producing a pharmaceutical composition for in vivo and in vitro gene therapy in mammals including humans. Furthermore, in some embodiments, the retroviral vector is especially replication-defective. They are used for delivering a polynucleotide in a non-integration manner.

Polynucleotides

In one aspect, provided is an RNA comprising, or alternatively consisting essentially of, or yet further consisting of: (a) a polynucleotide sequence of interest (SOI), (b) a U5 at the 3′ side of (a), and (c) a primer binding site (PBS) at the 3′ side of (b). In one aspect, the RNA is an isolated and/or engineered RNA. In one embodiment, the RNA further comprises one or more of the following: an encapsidation signal (Psi), a coding sequence for a gag gene, a coding sequence for a pol gene, a coding sequence for an env gene, an RNA sequence of a lentiviral tat gene, an RNA sequence of a lentiviral rev gene, an RNA sequence of a lentiviral vif gene, an RNA sequence of a lentiviral vpr gene, an RNA sequence of a lentiviral vpu gene, an RNA sequence of a lentiviral nef gene, or an RNA sequence of another retroviral or lentiviral accessory gene(s); a polypurine tract sequence (PPT), a central PPT (cPPT), a Rev-Responsive Element (RRE), an RNA sequence corresponding to a Woodchuck Hepatitis Virus (WHP) Posttranscriptional Regulatory Element (WPRE), and a U3 at the 3′ side of (c); the endogenous R region or a heterologous polyadenylation (pA) signal at the 3′ side of the downstream U3, an internal ribosome entry site (IRES), or a coding sequence encoding a protein. In one aspect, the RNA is isolated or recombinant.

In some embodiments, the pA signal is a strong exogenous pA signal, optionally replacing an R region at the 3′ side of PBS. In one embodiment, the pA signal replaces the R region of the 3′ LTR and/or the SOI replaces the R region of the 5′ LTR in a retroviral or lentiviral vector and/or particle, and resulting in an RNA, vector, particle, or other embodiments and/or aspects as disclosed herein. Without wishing to be bound by the theory, with both R regions gone, the likelihood of recombination during transfection rederiving the upstream R region is minimized. Additionally or alternatively, as the natural viral pA signal generally allows a great deal of read-through transcription, leading to decreased titers from significant numbers of genomic copies failing to be packaged, replacement of the viral pA signal with a strong exogenous pA signal will maximize the number of packaged genomes (and thus increased titers). In one embodiment, the strong pA signal has a biological function, e.g., in mediating the addition of a stretch of polyadenine to the 3′end of the mRNA, at a level similar to a simian virus 40 (SV40) pA signal, including but not limited to at least about 50%, or at least about 60%, or at least about 70%, or at least about 80%, or at least about 90%, or at least about 95%, or at least about 100%, or more than about 1 fold of SV40 pA signal. In some embodiments, the pA signal is selected from the group of a simian virus 40 (SV40) pA signal, a bovine growth hormone (BGH) pA signal, or a thymidine kinase (TK) pA signal.

In some embodiments, the SOI is heterologous to one or more of the other components of the RNA, for example, those components from or derived from a retrovirus (such as a lentivirus). In one embodiment, the SOI is not from or derived from a retrovirus (such as a lentivirus). In another embodiment, the SOI is heterologous to one or more of: the U5 of (b), the PBS of (c), the Psi, the gag gene, the pol gene, the env gene, the PPT, or the U3 at the 3' side of (c). Additionally or alternatively, the SOI is heterologous to any one or more of the following: the pA signal, the IRES, the coding sequence encoding a protein, or the protein encoded by the coding sequence. In one embodiment, any one or more of the following: the pA signal, the IRES, the coding sequence encoding a protein, or the protein encoded by the coding sequence, is heterologous to one or more of: the U5 of (b), the PBS of (c), the Psi, the gag gene, the pol gene, the env gene, the PPT, or the U3 at the 3′ side of (c). In a further embodiment, any one or more of the following: the pA signal, the IRES, the coding sequence encoding a protein, or the protein encoded by the coding sequence, is heterologous to a retrovirus (such as a lentivirus).

In some embodiments, the SOI comprises or consists essentially of, or yet further consists of an antisense strand of any one or more of: a donor template polynucleotide, or an antisense oligonucleotide (ASO). Additionally or alternatively, the SOI comprises or consists essentially of, or yet further consists of any one or more of: a micro RNA, a small interfering RNA (siRNA), a messenger RNA (mRNA), a guide polynucleotide, or an inhibitory RNA. In one embodiment, the donor template polynucleotide serves as a template in the process of homologous recombination (optionally caused by a CRISPR system) and that carries the modification that is to be introduced into a target sequence in a cell, optionally thereby correcting one or more mutations (such as those causing a genetic disease or condition) to its non-pathological wildtype nucleotide residue(s). In another embodiment, the inhibitory RNA, and/or micro RNA, and/or siRNA, and/or ASO interfere with a pathological gene expression and/or a pathological RNA (such as a mRNA). Such pathological gene expression or RNA may result in a genetic disease or condition. In yet another embodiment, the mRNA of the SOI expresses a gene product (such as a polypeptide) that treats a disease (such as a genetic disease). In one embodiment, the SOI comprises or consists essentially of, or yet further consists of, or acts as a vaccine adjuvant.

In some embodiments, the SOI comprises, or consists essentially of, or yet further consists of one or more RNA sequences, each of which is complementary to either strand of a cloning site. In one embodiment, the SOI comprises, or consists essentially of, or yet further consists of RNA sequence complementary to either strand of a multiple cloning site (MCS). In one embodiment, the cloning sites are the same or different. In a further embodiment, the cloning sites can be recognized and cut by the same or different restriction enzyme(s).

In some embodiments, the SOI comprises, or consists essentially of, or yet further consists of one or more pairs of RNA sequences, each pair of which comprises, or consists essentially of, or yet further consists of two RNA sequences complementary to either strand of a recombineering site. In one embodiment, the recombineering sites are the same and/or different.

In some embodiments, the SOI comprises one or more RNA sequences, each of which is complementary to either strand of a double-strand DNA which can be recognized and cut by a restriction enzyme (i.e. either strand of a cloning site). In some embodiments, the SOI and/or the RNA lack an R region.

In some embodiments, the SOI comprises or consists essentially of, or yet further consists of more than one (such as 2, or 3, or 4, or 5, or 6, or 7, or 8, or 9, or 10) of: an antisense strand of a donor template polynucleotide, a micro RNA, a small interfering RNA (siRNA), a messenger RNA (mRNA), a guide polynucleotide, an inhibitory RNA, an antisense strand of an antisense oligonucleotide (ASO), or any heterologous polynucleotide. In yet another embodiment, the more than one components of the SOI are separated by endonuclease site that can be recognized and cleaved by a endonuclease.

In some embodiments, the RNA comprises or consists essentially of, or yet further consists of the following, optionally from 5′ to 3′: (a) a SOI, (b) a U5, (c) a PBS, (d) a Psi, (e) an optional IRES, (f) an optional coding sequence encoding a protein and/or one or more RNA sequences, each of which is complementary to either strand of a cloning site (such as a multiple cloning site) and/or a recombineering site, (g) a PPT, (h) a U3, and (i) the endogenous R region or an optional pA signal.

In some embodiments, the encoded protein of (f) comprises or consists essentially of, or yet further consists of a polypeptide/protein component of an RNA interference (RNAi), CRISPR, TALEN, ZFN or other systems that target specific sequences to reduce or eliminate expression and/or function of proteins. In a further embodiment, the encoded protein of (f) comprises or consists essentially of, or yet further consists of a polynucleotide that expresses a Cas enzyme, a TALEN, a ZFN or other genome editing enzymes. In yet a further embodiment, the SOI of (a) comprises or consists essentially of, or yet further consists of a polynucleotide component of an RNA interference (RNAi), CRISPR, TALEN, ZFN or other systems that target specific sequences to reduce or eliminate expression and/or function of proteins, such as a sense strand or an antisense strand of a donor template polynucleotide and/or a guide polynucleotide.

In some embodiments, the RNA further comprises a coding sequence encoding a protein. In one embodiment, the coding sequence is a part of or the sense strand of the SOL In another embodiment, the coding sequence is between a Psi and a PPT or U3. In a further embodiment, an IRES is located in the RNA, optionally at the 5′ side of the coding sequence. Additionally or alternatively, the protein encoded by the coding sequence comprises or consists essentially of, or yet further consists of a clustered regularly interspaced short palindromic repeats (CRISPR) associated (Cas) enzyme. In one embodiment, the protein encoded by the coding sequence comprises or consists essentially of, or yet further consists of one or more of the following: a Cas enzyme, a TALEN, a ZFN or other genome editing enzymes. In a further embodiment, the SOI comprises a sense or antisense strand of a donor template polynucleotide suitable for use in a CRISPR system.

In some embodiments, the SOI comprises or consists essentially of, or yet further consists of, optionally from 5′ to 3′, (a1) a sense or antisense strand of a donor template polynucleotide suitable for use in a CRISPR system, (a2) an IRES and (a3) a coding sequence encoding a Cas enzyme.

In some embodiments, the Cas enzyme is selected from: Cash, Cas9, Cas12a (Cpf1), Cas13, or a variant of each thereof.

In some embodiments, non-limiting examples of the coding sequences (such as a mRNA) are one or more elements of the group consisting of marker genes, therapeutic genes, antiviral genes, antitumour genes, cytokine genes and/or toxin genes. such marker and therapeutic genes are preferably selected from one or more elements of the group consisting of β-galactosidase gene, neomycin gene, Herpes Simplex Virus thymidine kinase gene, puromycin gene, cytosine deaminase gene, hygromycin gene, secreted alkaline phosphatase gene, guanine phosphoribosyl transferase (gpt) gene, alcohol dehydrogenase gene, hypoxanthine phosphoribosyl transferase (HPRT) gene, green fluorescent protein (gfp) gene, cytochrome P450 gene and/or toxin genes such as a subunit of diptheria, pertussis toxin, tetanus toxoid.

In some embodiments, the SOI is about 10 nucleotides (nt) long to about 10⁶ nt. In one embodiment, the SOI is no more than about 10⁵ nt. In another embodiment, the SOI is no more than about 10⁴ nt. Additionally or alternatively, the SOI is more than about 10 nt long, or alternatively more than about 20 nt long, or alternatively more than about 30 nt long, or alternatively more than about 40 nt long, or alternatively more than about 50 nt long, or alternatively more than about 60 nt long, or alternatively more than about 70 nt long, or alternatively more than about 80 nt long, or alternatively more than about 90 nt long, or alternatively more than about 100 nt long, or alternatively more than about 200 nt long, or alternatively more than about 300 nt long, or alternatively more than about 400 nt long, or alternatively more than about 500 nt long, or alternatively more than about 600 nt long, or alternatively more than about 700 nt long, or alternatively more than about 800 nt long, or alternatively more than about 900 nt long, or alternatively more than about 1000 nt long. In one embodiment, the SOI is about 100 nt to about 1000 nt long. In another embodiment, the SOI is about 1000 nt to about 10000 nt long, for example, about 1000 nt to about 2000 nt, about 2000 nt to about 3000 nt, about 3000 nt to about 4000 nt, about 4000 nt to about 5000 nt, about 5000 nt to about 6000 nt, about 6000 nt to about 7000 nt, about 7000 nt to about 8000 nt, about 8000 nt to about 9000 nt, about 9000 nt to about 10000 nt long. In one embodiment, the SOI is about 50 nt long. In another embodiment, the SOI is about 500 nt long. In yet another embodiment, the SOI is about 5000 nt long.

In some embodiments, any of the non-SOI components is from or derived from a virus in the Retroviridae family. In one embodiment, any one or more of the following is from or derived from a virus in the Retroviridae family: U5, PBS, Psi, gag gene, pol gene, env gene, PPT, or U3. In some embodiments, the virus is in the Orthoretrovirinae subfamily or the Spumaretrovirinae subfamily of the Retroviridae family. In some embodiments, the virus is selected from Alpharetrovirus, Betaretrovirus, Deltaretrovirus, Epsilonretrovirus, Gammaretrovirus, or

Lentivirus. In one embodiment, the Alpharetrovirus is selected from Avian carcinoma Mill Hill virus 2, Avian leukosis virus (ALV), Avian myeloblastosis virus, Avian myelocytomatosis virus 29, Avian sarcoma leukosis virus (ASLV), Avian sarcoma virus CT10, Fujinami sarcoma virus, Rous sarcoma virus, UR2 sarcoma virus, or Y73 sarcoma virus. In another embodiment, the Betaretrovirus is selected from Langur virus, Mason-Pfizer monkey virus (MPMV), Mouse mammary tumor virus (MMTV), Squirrel monkey retrovirus, or Jaagsiekte sheep retrovirus. In yet another embodiment, the Deltaretrovirus is selected from Human T-lymphotropic virus (also named Human T-cell Leukaemia Virus, optionally selected from HTLV-1, HTLV-2, HTLV-3, HTLV-4), adult T-cell leukemia virus (ATLV), Simian-T-lymphotropic virus (types 1-4), Primate T-lymphotropic virus 1, Primate T-lymphotropic virus 2, Primate T-lymphotropic virus 3, or Bovine leukemia virus (BLV). In a further embodiment, the Epsilonretrovirus is selected from Walleye dermal sarcoma virus, Walleye epidermal hyperplasia virus 1, or Walleye epidermal hyperplasia virus 2; wherein the Gammaretrovirus is selected from Chick syncytial virus, Murine Sarcoma Virus (MSV), Finkel-Biskis-Jinkins murine sarcoma virus, Gardner-Arnstein feline, sarcoma virus, Gibbon ape leukemia virus, Guinea pig type-C oncovirus, Hardy-Zuckerman, feline sarcoma virus, Harvey murine sarcoma virus, Kirsten murine sarcoma virus, Moloney murine sarcoma virus, Porcine type-C oncovirus, Reticuloendotheliosis virus, Snyder-Theilen feline sarcoma virus, Trager duck spleen necrosis virus, Viper retrovirus, Woolly monkey sarcoma virus, Murine leukemia virus (MLV), Abelson murine leukemia virus, Friend virus, Feline leukemia virus (FELV), Koala retrovirus (KoRV), or Xenotropic murine leukemia virus-related virus. In yet a further embodiment, the Lentivirus is selected from human immunodeficiency virus (HIV), human immunodeficiency virus 1, human immunodeficiency virus 2, Simian immunodeficiency virus (SIV), Feline immunodeficiency virus (FIV), Puma lentivirus (PLV), Equine infectious anemia virus (EIAV), Bovine immunodeficiency virus (BIV), Caprine arthritis encephalitis virus, Jembrana disease virus, or Visna-maedi virus. In some embodiment, the virus is selected from the Spumaretrovirinae subfamily, optionally consisting of Bovispumavirus, Equispumavirus, Felispumavirus, Prosimiispumavirus, or Simiispumavirus. In one embodiment, the virus is selected from Simian foamy virus or Human foamy virus.

In some embodiment, the RNA lacks a R region. In one embodiment, the RNA lacks a 5′ R region or a 3′ R region or both.

In some embodiment, the RNA lacks a segment thereof that performs as a functional R region. In one embodiment, the RNA lacks a segment of the RNA that is located at the 5′ side of the U5 of (b) and shares at least 90% identity (including at least about 91% identity, or at least about 92% identity, or at least about 93% identity, or at least about 94% identity, or at least about 95% identity, or at least about 96% identity, or at least about 97% identity, or at least about 98% identity, or at least about 929 identity, or up to about 100% identity) to a segment of the RNA located at the 3′ side of the PBS of (c). In a further embodiment, the segment is about 1 nt long to about 500 nt long, including about 1 nt long to about 100 nt long. In yet a further embodiment, the segment is longer than about 10 nt, or about 20 nt, or about 30 nt, or about 40 nt, or about 50 nt, or about 60 nt, or about 70 nt, or about 80 nt, or about 90 nt, or about 100 nt, or about 200 nt, or about 300 nt, or about 400 nt. Additionally or alternatively, the segment is no more than about 500 nt, or no more than about 400 nt, or no more than about 300 nt, or no more than about 200 nt, or no more than about 100 nt, or no more than about 90 nt, or no more than about 80 nt, or no more than about 70 nt, or no more than about 60 nt, or no more than about 50 nt, or no more than about 40 nt, or no more than about 30 nt, or no more than about 20 nt, or no more than about 10 nt.

In one embodiment, the RNA further comprises a detectable or selection marker.

In another aspect, provided is a polynucleotide complementary to or corresponding to or encoding the RNA as disclosed herein. In one aspect, the polynucleotide is an isolated and/or engineered polynucleotide. In one embodiment, the polynucleotide further comprises a detectable or selection marker. Additionally or alternatively, the polynucleotide is selected from the group consisting of a single-strand DNA, a single-strand RNA, or a single-strand polynucleotide comprising both deoxyribonucleotide residue(s) and ribonucleotide residue(s).

In some embodiments, the polynucleotide complementary to or corresponding to or encoding the RNA comprises or consists essentially of, or yet further consists of one or more cloning sites and/or either strand of the one or more cloning sites. In some embodiments, the polynucleotide complementary to or corresponding to or encoding the RNA comprises or consists essentially of, or yet further consists of a multiple cloning site (MCS) and/or either strand of the MCS. In some embodiments, the cloning site is suitable for being cleaved by a restriction enzyme and leaving two sticky-end overhangs. In some embodiments, the cloning site is suitable for being cleaved (optionally by a restriction enzyme) and leaving two blunt ends. In one embodiment, the cloning sites are the same or different. In one embodiment, the cloning sites can be recognized and cut by the same or different restriction enzymes.

In some embodiments, the polynucleotide comprises one or more recombineering sites and/or either strand of the one or more recombineering sites. In some embodiments, the recombineering sites or consists essentially of, or yet further consists of same or different pairs of homology arms.

Such cloning site, overhangs, blunt ends, and/or recombineering sites allow insertion of a further polynucleotide (for example one with complementary overhangs, or with blunt ends, or flanked by a pair of homology arms of the recombineering site) in to the polynucleotide complementary to or corresponding to or encoding the RNA, thus permitting one of skill in the art to tailor the SOI, the RNA, the polynucleotide and/or other embodiments/aspects as disclosed herein for his/her use.

In some embodiments, the SOI and/or its corresponding DNA is in the polynucleotide in one or the other transcriptional orientation.

In yet another aspect, provided is a polynucleotide encoding the RNA as disclosed herein. In one aspect, the polynucleotide is an isolated and/or engineered polynucleotide. In one embodiment, the polynucleotide further comprises a detectable or selection marker. Additionally or alternatively, the polynucleotide is selected from the group consisting of a single-strand DNA, a single-strand RNA, or a single-strand polynucleotide comprising both deoxyribonucleotide residue(s) and ribonucleotide residue(s). In one embodiment, the polynucleotide further comprises a regulatory sequence that controls the transcription to the RNA. In a further embodiment, the regulatory sequence comprises or consists essentially of, or yet further consists of one or more of the sequences selected from the group consisting of: a promoter, a U3, an enhancer, an intron, a TATA box, an insulator, a silencer, 5′ cap, a polyadenylation sequence encoding a pA signal, a Woodchuck Hepatitis Virus (WHP) Posttranscriptional Regulatory Element (WPRE), a sequence encoding an IRES, or a signal sequence (for example, a coding sequence encoding a signal peptide).

In some embodiments, the promoter is a promoter (Pro) heterologous to one or more of the other components of the polynucleotide or any non-SOI component of the RNA encoded by the polynucleotide. In one embodiment, the promoter is heterologous to any one or more of the following: the U5 of (b), the PBS of (c), the Psi, the gag gene, the pol gene, the env gene, a lentiviral tat gene, a lentiviral rev gene, a lentiviral vif gene, a lentiviral vpr gene, a lentiviral vpu gene, a lentiviral nef gene, or another retroviral or lentiviral accessory gene(s), the PPT, or the U3 at the 3′ side of (c). Additionally or alternatively, the promoter is heterologous to any one or more of the following: the SOI, the pA signal, the IRES, the coding sequence encoding a protein, one or more RNA sequences, each of which is complementary to either strand of a cloning site (such as a multiple cloning site) and/or a recombineering site, one or more of cloning sites or either strand thereof, one or more of recombineering sites or either strand thereof, or the protein encoded by the coding sequence. In one embodiment, the promoter is suitable for use in a cell, such as a eukaryotic cell. In one embodiment, the promoter is selected from the group of a cytomegalovirus immediate-early promoter (CMV), a simian virus 40 early promoter (SV40), or a Rous sarcoma virus LTR promoter (RSV). In one embodiment, encoding the protein by the coding sequence is driven by an IRES. In a further embodiment, the IRES and a coding sequence encoding a protein is at the 3′ side of PBS, i.e., the traditional multi-cloning site of a retroviral vector. Without wishing to be bound by the theory, the expression is low and transient, but it may be ideal for expression of relevant nucleases or other proteins.

Additionally provided is a polynucleotide delivered to a cell or a subject via a method as described herein.

In one aspect, provided is a polynucleotide complementary to or corresponding to any polynucleotide as described above.

Also provided is a polynucleotide comprising, or alternatively consisting essentially of, or yet further consisting of one or more of the following: an RNA as described herein, or a polynucleotide as described herein.

In some embodiments, the polynucleotide as described herein is selected from the group consisting of a single-strand DNA, a single-strand RNA, a single-strand polynucleotide comprising both deoxyribonucleotide residue(s) and ribonucleotide residue(s), a double-strand DNA, a double-strand RNA, a DNA/RNA hybrid, or any combination thereof.

In one aspect, provided is a provirus comprising, or alternatively consisting essentially of, or yet further consisting of one or more of the following: an RNA as described herein, or a polynucleotide as described herein. Also provided is a messenger RNA (mRNA) of the provirus as described herein.

In another aspect, provided is an RNA comprising, or alternatively consisting essentially of, or yet further consisting of a genomic RNA of a viral particle as disclosed herein.

Vectors and Viral Particles

In one aspect, provided is a vector comprising one or more of the following: a RNA as described herein, or a polynucleotide as described herein. In some embodiments, the vector is a non-viral vector. In one embodiment, the non-viral vector is selected from plasmids, inorganic particles, calcium phosphate particles, silica nanoparticles, gold nanoparticles, nanoparticles, cationic lipids, lipid nano emulsions, solid lipid nanoparticles, peptide based vectors, polymer based vectors, liposomes, or gelatin-based vectors. In some embodiments, the vector is a viral vector. In one embodiment, the viral vector is selected from the group of a retroviral vector, a lentiviral vector, an adenoviral vector, or an adeno-associated viral vector.

In another aspect, provided is a viral particle comprising an RNA as described herein.

In some embodiments, the viral particle is a retroviral particle (such as a gammaretroviral particle or a lentiviral particle). In some embodiments, the viral particle is in the Retroviridae family. In one embodiment, the viral particle is in the Orthoretrovirinae subfamily or the Spumaretrovirinae subfamily of the Retroviridae family. In some embodiments, the viral particle is selected from Alpharetrovirus, Betaretrovirus, Deltaretrovirus, Epsilonretrovirus, Gammaretrovirus, or Lentivirus. In one embodiment, the Alpharetrovirus is selected from Avian carcinoma Mill Hill virus 2, Avian leukosis virus (ALV), Avian myeloblastosis virus, Avian myelocytomatosis virus 29, Avian sarcoma leukosis virus (ASLV), Avian sarcoma virus CT10, Fujinami sarcoma virus, Rous sarcoma virus, UR2 sarcoma virus, or Y73 sarcoma virus. In another embodiment, the Betaretrovirus is selected from Langur virus, Mason-Pfizer monkey virus (MPMV), Mouse mammary tumor virus (MMTV), Squirrel monkey retrovirus, or Jaagsiekte sheep retrovirus. In yet another embodiment, the Deltaretrovirus is selected from Human T-lymphotropic virus (also named Human T-cell Leukaemia Virus, optionally selected from HTLV-1, HTLV-2, HTLV-3, HTLV-4), adult T-cell leukemia virus (ATLV), Simian-T-lymphotropic virus (types 1-4), Primate T-lymphotropic virus 1, Primate T-lymphotropic virus 2, Primate T-lymphotropic virus 3, or Bovine leukemia virus (BLV). In a further embodiment, the Epsilonretrovirus is selected from Walleye dermal sarcoma virus, Walleye epidermal hyperplasia virus 1, or Walleye epidermal hyperplasia virus 2; wherein the Gammaretrovirus is selected from Chick syncytial virus, Murine Sarcoma Virus (MSV), Finkel-Biskis-Jinkins murine sarcoma virus, Gardner-Arnstein feline, sarcoma virus, Gibbon ape leukemia virus, Guinea pig type-C oncovirus, Hardy-Zuckerman, feline sarcoma virus, Harvey murine sarcoma virus, Kirsten murine sarcoma virus, Moloney murine sarcoma virus, Porcine type-C oncovirus, Reticuloendotheliosis virus, Snyder-Theilen feline sarcoma virus, Trager duck spleen necrosis virus, Viper retrovirus, Woolly monkey sarcoma virus, Murine leukemia virus (MLV), Abelson murine leukemia virus, Friend virus, Feline leukemia virus (FELV), Koala retrovirus (KoRV), or Xenotropic murine leukemia virus-related virus. In yet a further embodiment, the Lentivirus is selected from human immunodeficiency virus (HIV), human immunodeficiency virus 1, human immunodeficiency virus 2, Simian immunodeficiency virus (SIV), Feline immunodeficiency virus (FIV), Puma lentivirus (PLV), Equine infectious anemia virus (EIAV), Bovine immunodeficiency virus (BIV), Caprine arthritis encephalitis virus, Jembrana disease virus, or Visna-maedi virus. In some embodiment, the viral particle is selected from the Spumaretrovirinae subfamily, optionally consisting of Bovispumavirus, Equispumavirus, Felispumavirus, Prosimiispumavirus, or Simiispumavirus. In one embodiment, the viral particle is selected from Simian foamy virus or Human foamy virus.

In some embodiments, the viral particle further comprises one or more of the following: a protein encoded by a gag gene (such as a group-specific antigen precursor polyprotein and/or its processed group-specific antigen polyprotein(s) including but not limited to a nucleocapsid (NC), a capsid protein (CA) or a matrix protein (MA)), a protein encoded by a pol gene (such as a precursor polyprotein encoded by a pol gene and/or its processed polyprotein(s) including but not limited to a reverse transcriptase (RT), an RNase H domain optionally as part of a RT or any other polypeptide, an integrase (IN), or a protease (PR)), a protein encoded by an env gene (i.e., an envelope protein, such as a precursor polyprotein encoded by an env gene and/or its processed polyprotein(s) including but not limited to a surface envelope protein and a transmembrane envelope protein), a protein encoded by a lentiviral tat gene, a protein encoded by a lentiviral rev gene, a protein encoded by a lentiviral vif gene, a protein encoded by a lentiviral vpr gene, a protein encoded by a lentiviral vpu gene, a protein encoded by a lentiviral nef gene, or a protein encoded by another retroviral or lentiviral accessory gene(s).

In some embodiments, the viral particle further comprises an endonuclease or a polynucleotide encoding an endonuclease.

In some embodiments, the viral particle further comprises a detectable or selection marker.

In some embodiments, the viral particle further comprises a lipid bilayer, for example, packaging the polynucleotide and/or RNA therein. In some embodiments, the lipid bilayer further comprises a protein encoded by an env gene.

In some embodiments, the RNase H domain is a wild type RNase H. In some embodiments, the RNase H domain comprises a mutated RNase H defective in degrading RNA. In one embodiment, the RNase H comprises a Y586F mutant in a gammaretrovirus (RV) RNase H domain. In one embodiment, the RNase H domain comprises one or more of the following mutations, for example in a RV RNase H domain: Y586F, D524N, Δ5E, ΔC, and H7. In another embodiment, the RNase H domain comprises E478Q, for example in an LV RNase H domain.

In one embodiments, Y586F, D524N and E478Q refer to point mutations in the RNase H domain naming by appending the amino acid present in wild-type RT, the residue number, and the amino acid present in the mutant at that position. In one embodiment, Δ5E refers to a 5-amino acid deletion from Ser-643 to Arg-647. In one embodiment, AC refers to an 11-amino acid deletion from Ile-593 through Leu-603 in the RNase H domain. In one embodiment, H7 refers to a linker insertion mutant, containing a frameshift at the start of the RNase H domain, and thus is effectively an RNase H null form of RT. In one embodiment, any one or more of the mutations renders a RT or a protein comprising the RNase H domain defective in degrading RNA.

In some embodiments, the RT is a wild type RT. In some embodiments, the RT comprises or consists essentially of, or yet further consists of a mutated RT defective in degrading RNA and/or mediating strand transfer. Without wishing to be bound by the theory, those mutants reduce or eliminate any chance of the nascent single strand (ss) DNA undergoing a strong stop jump to an area of microhomology, either viral or cellular. In one embodiment, the RT comprises one or more of the following mutations, for example in a RV RNase H domain of the RT: Y586F, D524N, Δ5E, 4C, and H7. In another embodiment, the RT comprises E478Q, for example in an LV RNase H domain of the RT. In one embodiment, the RT comprises a L92P mutant and/or a F61A of a lentivirus (LV) RT. In another embodiment, the RT comprises a Y598V mutant of a retrovirus (RV) RT. Additionally or alternatively, the RT is a high fidelity reverse transcriptase, for example via comprising the following any one or two or all three mutations: W229A and V75I and K65R in an LV RT.

In one embodiment, L92P, F61A and Y598V refer to point mutations in the RT naming by appending the amino acid present in wild-type RT, the residue number, and the amino acid present in the mutant at that position. In one embodiment, any one or both of the mutations renders a RT defective in mediating strand transfer.

In some embodiments, the NC is a wildtype NC or a mutant thereof defective in mediating strand transfer. In some embodiments, the NC is a wildtype NC. In some embodiments, the NC is a mutated NC defective in mediating strand transfer.

In some embodiments, the integrase is a wildtype integrase. In some embodiments, the integrase is defective in integrating a polynucleotide into a chromosomal DNA. In a further embodiment, the integrase comprises a D64V mutation in an LV integrase.

In some embodiments, the envelope protein is an amphotropic envelope protein, an ecotropic envelope protein, or an xenotropic envelope protein. Additionally or alternatively, the envelope protein is from or derived from 10A1 Murine Leukemia Virus (MuLV) envelopes, Gibbon-ape leukemia virus (GaLV) envelopes, Vesicular stomatitis virus G (VSV-G) envelopes, and/or FeLVB envelopes.

In some embodiments, the viral particle as described herein comprising, or alternatively consisting essentially of, or yet further consisting of the following: a vector genome comprising an RNA as described herein, a capsid comprising, or alternatively consisting essentially of, or yet further consisting of a capsid protein (CA) encoded by a gag gene and/or a matrix protein (MA) encoded by a gag gene, a lipid bilayer further comprising an envelope protein encoded by an env gene, an RT encoded by a pol gene, wherein the RT is a L92P mutant and/or a F61A of a lentivirus (LV) or a Y598V mutant of a retrovirus (RV) RT, a nucleocapsid (NC), and an optional integrase that is defective in integrating a polynucleotide into a chromosomal DNA.

In some embodiments, the viral particle as described herein comprising, or alternatively consisting essentially of, or yet further consisting of the following: a vector genome comprising an RNA as described herein, a capsid comprising, or alternatively consisting essentially of, or yet further consisting of a capsid protein (CA) encoded by a gag gene and/or a matrix protein (MA) encoded by a gag gene, a lipid bilayer further comprising an envelope protein encoded by an env gene, an RNase H encoded by a pol gene, wherein the RNase H is a Y586F, D524N, Δ5E, 4C, and/or H7 mutant in a gammaretrovirus (RV) RNase H or a E478Q mutant in a lentivirus (LV) RNase H domain, a nucleocapsid (NC), and an optional integrase that is defective in integrating a polynucleotide into a chromosomal DNA.

In some embodiments, the viral particle as described herein comprising, or alternatively consisting essentially of, or yet further consisting of the following: a vector genome comprising the engineered RNA as described herein, a capsid comprising, or alternatively consisting essentially of, or yet further consisting of a capsid protein (CA) encoded by a gag gene and/or a matrix protein (MA) encoded by a gag gene, a lipid bilayer further comprising an envelope protein encoded by an env gene, an RNase H encoded by a pol gene, a nucleocapsid (NC), wherein the NC is a mutated NC defective in mediating strand transfer, and an optional integrase that is defective in integrating a polynucleotide into a chromosomal DNA.

As it is understood, any one of the embodiments can be combined with another. For example, the vector and/or particle may comprise a RT that has a high-fidelity mutation and is defective in mediating a strand transfer and an integrase with a mutation that eliminates its activity.

Also provided is a retroviral or lentiviral particle produced by a method as described herein.

In one embodiment, the vector and/or particles as disclosed herein may be reduced in titer to some degree, but are still sufficient in transducing and/or transfecting a cell and/or a subject.

Cells

In one aspect, provided is a cell comprising one or more of the following: an RNA of as described herein, a polynucleotide as described herein, a vector as described herein, or a particle as described herein. In one embodiment, the cell is a eukaryotic cell or a prokaryotic cell.

In some embodiments, the cell is derived from a packaging cell line optionally selected from the group consisting of psi-2, psi-Crypt, psi-AM, GP+E-86, PA317, GP+envAM-12, Fly A13, BOSC 23, BING, Fly RD 18, ProPak-X, -A.52 and -A.6.

In some embodiments, the cell further comprises or expresses one or more of the following: a protein encoded by a gag gene (such as a group-specific antigen precursor polyprotein and/or its processed group-specific antigen polyprotein(s) including but not limited to a nucleocapsid (NC), a capsid protein (CA) or a matrix protein (MA)), a protein encoded by a pol gene (such as a precursor polyprotein encoded by a pol gene and/or its processed polyprotein(s) including but not limited to a reverse transcriptase (RT), an RNase H domain optionally as part of a RT or any other polypeptide, an integrase (IN), or a protease (PR)), a protein encoded by an env gene (such as a precursor polyprotein encoded by an env gene and/or its processed polyprotein(s) including but not limited to a surface envelope protein and a transmembrane envelope protein), a protein encoded by a lentiviral tat gene, a protein encoded by a lentiviral rev gene, a protein encoded by a lentiviral vif gene, a protein encoded by a lentiviral vpr gene, a protein encoded by a lentiviral vpu gene, a protein encoded by a lentiviral nef gene, a protein encoded by another retroviral or lentiviral accessory gene(s), a gag gene, a pol gene, an env gene, a lentiviral tat gene, a lentiviral rev gene, a lentiviral vif gene, a lentiviral vpr gene, a lentiviral vpu gene, a lentiviral nef gene, or another retroviral or lentiviral accessory gene(s).

In some embodiments, the cell further comprises one or more of the following: an endonuclease, a polynucleotide encoding an endonuclease, or a polynucleotide that is a reverse complement of the endonuclease-coding polynucleotide.

In some embodiments, the RNase H is a wild type RNase H. In some embodiments, the RNase H comprises a mutated RNase H defective in degrading RNA. In one embodiment, the RNase H comprises one or more of Y586F, D524N, Δ5E, ΔC, and/or H7 mutations in a gammaretrovirus (RV) RNase H, or a E478Q mutation in a lentivirus (LV) RNase H domain.

In some embodiments, the RT comprises a mutated RT defective in mediating strand transfer. In one embodiment, the RT comprises a L92P mutant and/or a F61A of a lentivirus (LV) RT. In another embodiment, the RT comprises a Y598V mutant of a retrovirus (RV) RT.

In some embodiments, the NC is a wildtype NC. In some embodiments, the NC is a mutated NC defective in mediating strand transfer.

In some embodiments, the integrase is a wildtype integrase. In some embodiments, the integrase is defective in integrating a polynucleotide into a chromosomal DNA.

In some embodiments, the cell comprises any one or any two or all three of the following: (a) an expression vector comprising a gag gene and a pol gene, (b) an expression vector comprising an env gene, or (c) a polynucleotide as described herein.

In some embodiments, the cell comprises any one or any two or all three of the following: (a) an expression vector comprising a gag gene and a pol gene, (b) an expression vector comprising an env gene, or (c) a vector as described herein.

Methods of Production

In one aspect provided is a method of producing a retroviral or lentiviral particle.

In some embodiment, the production method comprises or consists essentially of, or yet further consists of:

(a) culturing a cell comprising at least one of: an RNA as described herein, a polynucleotide as described herein, or a vector as described herein, wherein the cell comprise and/or expresses one or more of the following: a protein encoded by a gag gene, a protein encoded by a pol gene, a protein encoded by an env gene, a gag gene, a pol gene, an env gene, a protein encoded by a lentiviral tat gene, a protein encoded by a lentiviral rev gene, a protein encoded by a lentiviral vif gene, a protein encoded by a lentiviral vpr gene, a protein encoded by a lentiviral vpu gene, a protein encoded by a lentiviral nef gene, a protein encoded by another retroviral or lentiviral accessory gene(s), a lentiviral tat gene, a lentiviral rev gene, a lentiviral vif gene, a lentiviral vpr gene, a lentiviral vpu gene, a lentiviral nef gene and/or another retroviral or lentiviral accessory gene(s), and optionally wherein at least one of the protein(s) encoded by a gag gene is a nucleocapsid (NC));

(b) optionally collecting supernatant of the cell culture; and

(c) optionally isolating or purifying retroviral or lentiviral particles from the collected supernatant;

In some embodiment, the production method comprises or consists essentially of, or yet further consists of:

(a) culturing a cell as described herein, wherein the cell comprise and/or expresses one or more of the following: a protein encoded by a gag gene, a protein encoded by a pol gene, a protein encoded by an env gene, a gag gene, a pol gene, an env gene, a protein encoded by a lentiviral tat gene, a protein encoded by a lentiviral rev gene, a protein encoded by a lentiviral vif gene, a protein encoded by a lentiviral vpr gene, a protein encoded by a lentiviral vpu gene, a protein encoded by a lentiviral nef gene, a protein encoded by another retroviral or lentiviral accessory gene(s), a lentiviral tat gene, a lentiviral rev gene, a lentiviral vif gene, a lentiviral vpr gene, a lentiviral vpu gene, a lentiviral nef gene and/or another retroviral or lentiviral accessory gene(s), and optionally wherein at least one of the protein(s) encoded by a gag gene is a nucleocapsid (NC), and optionally wherein at least one of the protein(s) encoded by a gag gene is a nucleocapsid (NC);

(b) optionally collecting supernatant of the cell culture; and

(c) optionally isolating or purifying retroviral or lentiviral particles from the collected supernatant.

In some embodiments, the cell is cultured for about 48 hours to about 72 hours and intervals within 48 to 72 hours.

In some embodiments, the production method further comprises concentrating the isolated or purified retroviral or lentiviral particles.

In some embodiments, the cell further expresses an endonuclease. In some embodiments, the cell further comprises a polynucleotide encoding an endonuclease or a polynucleotide that is a reverse complement of the endonuclease-coding polynucleotide.

As it would be understood by one of skill in the art, in some embodiments, the cell may be a host cell or a production cell, which producing an RNA, a polynucleotide, a vector, or a retroviral particle as disclosed herein. In other embodiments, the cell may be a target cell to which a polynucleotide is delivered to, via a method as disclosed herein.

Methods of Uses

In one aspect, provided is a method of delivering a polynucleotide to a cell. In some embodiments, the delivery method comprises or consists essentially of, or yet further consists of contacting the cell with a viral particle (such as a retroviral particle or a lentiviral particle) as disclosed herein. In one embodiment, the cell is a eukaryotic cell or a prokaryotic cell.

In another aspect, provided is a method of delivering a polynucleotide to a subject. In some embodiments, the delivery method comprises or consists essentially of, or yet further consists of administering a viral particle (such as a retroviral particle or a lentiviral particle) as disclosed herein to the subject. In one embodiment, the delivered polynucleotide comprises the sequence of interest (SOI) or a polynucleotide which is a reverse complement of the SOI or both. In one embodiment, the subject is a mammal. In a further embodiment, the subject is a human.

In some embodiments relating to any other embodiments and/or aspect, any component, composition and/or methods as disclosed herein, the delivered and/or produced polynucleotide comprises the sequence of interest (SOI) or a polynucleotide which is a reverse complement of the SOI or both.

In yet another aspect, provided is a method of producing a polynucleotide. In some embodiments, the method comprises or consists essentially of, or yet further consists of (a) contacting a cell with a viral particle (such as a retroviral particle or a lentiviral particle) as disclosed herein, (b) culturing the cell, and (c) optionally isolating or purifying the polynucleotide produced by the cell. In one embodiment, the produced polynucleotide comprises the sequence of interest (SOI) or a polynucleotide that is a reverse complement of the SOI or both.

In some embodiments, a method as described herein further comprises concentrating the isolated or purified polynucleotide.

In some embodiments, the delivered or produced polynucleotide is not a double-stranded DNA. In one embodiment, the delivered or produced polynucleotide is a single-stranded DNA or an RNA-DNA hybrid. Additionally or alternatively, the delivered or produced polynucleotide is free of any retroviral or lentiviral sequence at the 5′ end or the 3′ end or both ends.

In some embodiments, the delivered or produced polynucleotide comprises one or more of the following: a donor template polynucleotide, a micro RNA, a small interfering RNA (siRNA), a guide polynucleotide, an inhibitory RNA, a messenger RNA (mRNA), or an antisense oligonucleotide (ASO).

In some embodiments, the delivery and/or production methods and/or compositions thereof may be used for treating a disease or condition (such as a genetic disease as disclosed herein). In one embodiment, the donor template polynucleotide may serve as a template in the process of homologous recombination (optionally caused by a CRISPR system) and that carries the modification that is to be introduced into a target sequence in a cell, optionally thereby correcting one or more mutations (such as those causing a genetic disease or condition) to its non-pathological wildtype nucleotide residue(s). In another embodiment, the inhibitory RNA, and/or micro RNA, and/or siRNA, and/or ASO may interfere with a pathological (i.e., disease-causing) gene expression and/or a pathological (i.e., disease-causing) RNA (such as a mRNA). Such pathological gene expression or RNA may result in a genetic disease or condition. In yet another embodiment, the SOI may express a gene product (such as a polypeptide and/or a polynucleotide) that treats a disease (such as a genetic disease). In still another embodiment, the SOI comprises or consists essentially of, or yet further consists of, or acts as a vaccine adjuvant.

In some embodiments, the viral particle comprises an endonuclease or a polynucleotide encoding an endonuclease. In one embodiment, the endonuclease cleaves the delivered or produced polynucleotide and releases one or more of the following: a donor template polynucleotide, a micro RNA, a small interfering RNA (siRNA), a guide polynucleotide, an inhibitory RNA, a messenger RNA (mRNA), or an antisense oligonucleotide (ASO). In one embodiment, the guide polynucleotide is a guide RNA (gRNA) suitable for use in a CRISPR system.

In some embodiment, the delivered or produced polynucleotide, which is also referred to herein as a vector payload, is protected from cellular nucleases by incorporation into, for example, the viral pre-integration complex.

In some embodiments, a delivery method as described herein further comprises delivering a guide polynucleotide to the cell or subject. In one embodiment, the guide polynucleotide is a guide RNA (gRNA), optionally suitable for use in a CRISPR system. In one embodiment, the guide polynucleotide is delivered in a non-viral or viral vector.

Compositions and Kits

In one aspect, provided is a composition comprising, or alternatively consisting essentially of, or yet further consisting of one or more of a polynucleotide, a RNA, a cell, and/or viral particle as disclosed herein and a carrier. In one embodiment, the carrier is a pharmaceutically acceptable carrier. In one embodiment, the composition further comprises a preservative or stabilizer. In some embodiments, the composition further comprises a guide polynucleotide. In one embodiment, the guide polynucleotide is a guide RNA (gRNA), optionally suitable for use in a CRISPR system. Additionally or alternatively, the guide polynucleotide is in a non-viral or viral vector, such as a viral particle as disclosed herein or a vector other than the viral particle as disclosed herein.

In some aspects, provided herein is a composition comprising, consisting essentially of, or consisting of the combination of an agent as provided herein, and at least one pharmaceutically acceptable excipient.

In another aspect, provided is a kit comprising, or alternatively consisting essentially of, or yet further consisting of one or more of the following: an RNA as described herein; a polynucleotide as described herein; a vector as described herein; a viral particle as described herein; a cell as described herein; an expression vector comprising one or more of: a gag gene, a pol gene, an env gene, a lentiviral tat gene, a lentiviral rev gene, a lentiviral vif gene, a lentiviral vpr gene, a lentiviral vpu gene, a lentiviral nef gene, or another retroviral or lentiviral accessory gene(s); an expression vector encoding proteins required for the RNA to be packaged in a particle; the composition as described herein, and an optional instruction for use.

The composition of the present disclosure can be administered by parenteral (e.g., intramuscular, intraperitoneal, intravenous, ICV, intracisternal injection or infusion, subcutaneous injection, or implant), oral, by inhalation spray nasal, vaginal, rectal, sublingual, urethral (e.g., urethral suppository) or topical routes of administration (e.g., gel, ointment, cream, aerosol, etc.) and can be formulated in suitable dosage unit formulations containing conventional non-toxic pharmaceutically acceptable carriers, adjuvants, excipients, and vehicles appropriate for each route of administration.

The pharmaceutical compositions can be conveniently presented in dosage unit form and can be prepared by any of the methods well known in the art of pharmacy. The pharmaceutical compositions can be, for example, prepared by uniformly and intimately bringing the agents provided herein into association with a liquid carrier, a finely divided solid carrier or both, and then, if necessary, shaping the product into the desired formulation. For example, pharmaceutical compositions of the present technology may take a form suitable for virtually any mode of administration, including, for example, topical, ocular, oral, buccal, systemic, nasal, injection, infusion, transdermal, rectal, and vaginal, or a form suitable for administration by inhalation or insufflation. Useful injectable preparations include sterile suspensions, solutions, or emulsions of the agents provided herein in aqueous or oily vehicles. The compositions may also contain formulating agents, such as suspending, stabilizing, and/or dispersing agents. The formulations for injection can be presented in unit dosage form, e.g., in ampules or in multidose containers, and may contain added preservatives. Alternatively, the injectable formulation can be provided in powder form for reconstitution with a suitable vehicle, including but not limited to sterile pyrogen free water, buffer, and dextrose solution, before use. To this end, the combination of agents provided herein can be dried by any art-known technique, such as lyophilization, and reconstituted prior to use.

### EXAMPLES

The following examples are included to demonstrate some embodiments of the disclosure. However, those of skill in the art should, in light of the present disclosure, appreciate that many changes can be made in the specific embodiments which are disclosed and still obtain a like or similar result without departing from the spirit and scope of the invention.

### Example 1

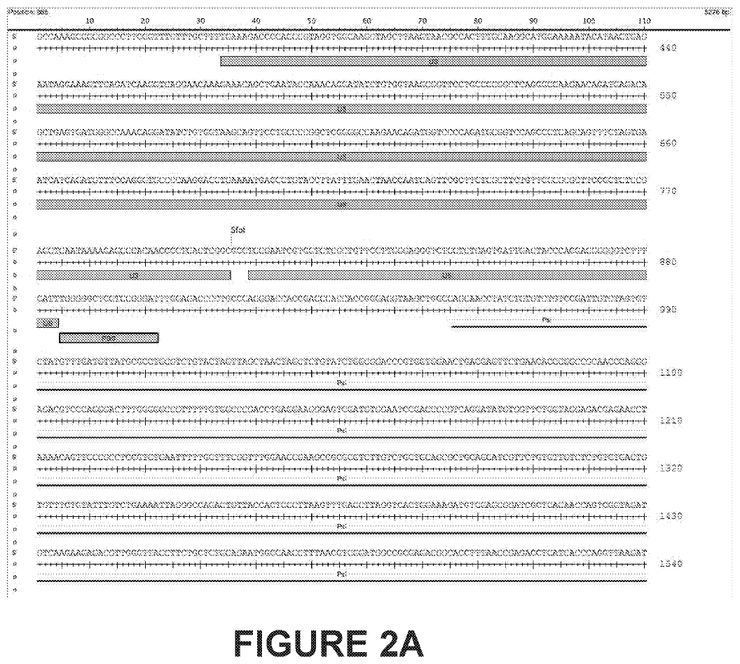

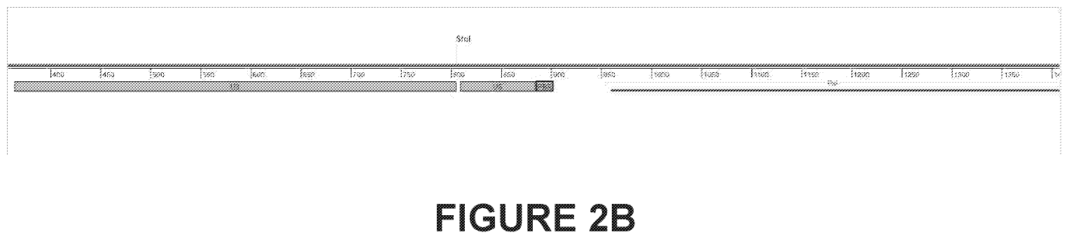

A polynucleotide comprising, from 5′ to 3′, a U3 region, a SOI comprising a cloning site but without an R region, a U5 region, a PBS, a Psi, a MCS, a PPT, a U3, and a pA was designed and constructed. The SOI of this polynucleotide comprises, or consists essentially of, or yet further consists of a sequence corresponding to one or both strand(s) of a cloning site. In one embodiment, such cloning site can be cut by the restriction enzyme, Sfol, as shown in FIG. 2A.

Also constructed was a vector (such as a plasmid) comprising a DNA sequence corresponding to the RNA (shown in FIG. 2B). This plasmid permits insertion of a further sequence, such as a donor template sequence, an ASO, or an antisense strand of a siRNA, microRNA or any other RNA as disclosed herein, thus producing an engineered RNA comprising, from 5′ to 3′, any SOI as disclosed herein, a U5 region, a PBS, a Psi a PPT, and a U3.

Such RNA is then packaged to a retroviral or lentiviral particle, and tested for transduction efficacy in vivo and in vitro.

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

It should be understood that although the present invention has been specifically disclosed by certain aspects, embodiments, and optional features, modification, improvement and variation of such aspects, embodiments, and optional features can be resorted to by those skilled in the art, and that such modifications, improvements and variations are considered to be within the scope of this disclosure.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

### REFERENCES

Adam, M. A., Osborne, W. R. A., & Miller, A. D. (1995). R-Region cDNA Inserts in Retroviral Vectors Are Compatible with Virus Replication and High-Level Protein Synthesis from the Insert. Human Gene Therapy, 6(9), 1169-1176.
Afonin, K. A., Desai, R., Viard, M., Kireeva, M. L., Bindewald, E., Case, C. L., Maciag, A. E., Kasprzak, W. K., Kim, T., Sappe, A., Stepler, M., Kewalramani, V. N., Kashlev, M., Blumenthal, R., . . . Shapiro, B. A. (2013). Co-transcriptional production of RNA-DNA hybrids for simultaneous release of multiple split functionalities. Nucleic acids research, 42(3), 2085-97.
Afonin, K. A., Viard, M., Martins, A. N., Lockett, S. J., Maciag, A. E., Freed, E. O., Heldman, E., Jaeger, L., Blumenthal, R., . . . Shapiro, B. A. (2013). Activation of different split functionalities on re-association of RNA-DNA hybrids. Nature nanotechnology, 8(4), 296-304.
Blain, S. W., & Goff, S. P. (1995). Effects on DNA synthesis and translocation caused by mutations in the RNase H domain of Moloney murine leukemia virus reverse transcriptase. Journal of virology, 69(7), 4440-4452.
Cheslock, S. R., Anderson, J. A., Hwang, C. K., Pathak, V. K., & Hu, W. S. (2000). Utilization of nonviral sequences for minus-strand DNA transfer and gene reconstitution during retroviral replication. Journal of virology, 74(20), 9571-9.
Gunzburg, W., Klein, D., Tabotta, W., Salmons, B. (2004). U.S. Pat. No. 6,821,776 B1. Washington, D.C.: US Patent and Trademark Office.
Herzig, E., Voronin, N., Kucherenko, N., & Hizi, A. (2015). A Novel Leu92 Mutant of HIV-1 Reverse Transcriptase with a Selective Deficiency in Strand Transfer Causes a Loss of Viral Replication. Journal of virology, 89(16), 8119-8129.
Keskin, H., Shen, Y., Huang, F., Patel, M., Yang, T., Ashley, K., Mazin, A. V., & Storici, F. (2014). Transcript-RNA-templated DNA recombination and repair. Nature, 515(7527), 436-9.

Ohle, C., Tesorero, R., Schermann, G., Dobrev, N., Sinning, I., & Fischer T. (2016). Transient RNA-DNA hybrids are required for efficient double-strand break repair. Cell, 167(4), 1001-1013.Sulej, A. A., Tuszynska, I., Skowronek, K. J., Nowotny, M., & Bujnicki, J. M. (2012). Sequence-specific cleavage of the RNA strand in DNA-RNA hybrids by the fusion of ribonuclease H with a zinc finger. Nucleic acids research, 40(22), 11563-11570.
Tanese, N., & Goff, S. P. (1988). Domain structure of the Moloney murine leukemia virus reverse transcriptase: mutational analysis and separate expression of the DNA polymerase and RNase H activities. Proceedings of the National Academy of Sciences of the United States of America, 85(6), 1777-1781.
Ci Y, Yang Y, Xu C, Shi L (2018). Vesicular stomatitis virus G protein transmembrane region is crucial for the hemi-fusion to full fusion transition. Sci Rep. 2018; 8(1):10669. Published 2018 Jul. 13. doi:10.1038/s41598-018-28868-y.
Sharan S K, Thomason L C, Kuznetsov S G, Court D L. Recombineering: a homologous recombination-based method of genetic engineering. Nat Protoc. 2009; 4(2):206-223.
Schatz, 0., Cromme, F. V., Gruninger-Leitch, F., & Le Grice, S. F. (1989). Point mutations in conserved amino acid residues within the C-terminal domain of HIV-1 reverse transcriptase specifically repress RNase H function. FEB S letters, 257(2), 311-314.
Halvas, E. K., Svarovskaia, E. S., & Pathak, V. K. (2000). Development of an in vivo assay to identify structural determinants in murine leukemia virus reverse transcriptase important for fidelity. Journal of virology, 74(1), 312-319.
Cherepanov, P., Pluymers, W., Claeys, A., Proost, P., De Clercq, E., & Debyser, Z. (2000). High-level expression of active HIV-1 integrase from a synthetic gene in human cells. FASEB journal: official publication of the Federation of American Societies for Experimental Biology, 14(10), 1389-1399.
Wisniewski, M., Palaniappan, C., Fu, Z., Le Grice, S. F., Fay, P., & Bambara, R. A. (1999). Mutations in the primer grip region of HIV reverse transcriptase can increase replication fidelity. The Journal of biological chemistry, 274(40), 28175-28184.
Rezende, L. F., Curr, K., Ueno, T., Mitsuya, H., & Prasad, V. R. (1998). The impact of multidideoxynucleoside resistance-conferring mutations in human immunodeficiency virus type 1 reverse transcriptase on polymerase fidelity and error specificity. Journal of virology, 72(4), 2890-2895.
Shah, F. S., Curr, K. A., Hamburgh, M. E., Parniak, M., Mitsuya, H., Arnez, J. G., & Prasad, V. R. (2000). Differential influence of nucleoside analog-resistance mutations K65R and L74V on the overall mutation rate and error specificity of human immunodeficiency virus type 1 reverse transcriptase. The Journal of biological chemistry, 275(35), 27037-27044.

What is claimed is:

1. A RNA comprising:
(a) a polynucleotide sequence of interest (SOI),
(b) a U5 at the 3′ side of (a), and
(c) a primer binding site (PBS) at the 3′ side of (b), wherein the RNA is optionally isolated or engineered.

2. The RNA of claim 1, wherein the RNA further comprises one or more of the following: an encapsidation signal (Psi), a coding sequence for a gag gene, a coding sequence for a pol gene, a coding sequence for an env gene, a polypurine tract sequence (PPT), a central PPT (cPPT), a Rev-Responsive Element (RRE), an RNA sequence of a Woodchuck Hepatitis Virus (WHP) Posttranscriptional Regulatory Element (WPRE), an RNA sequence of a lentiviral tat gene, an RNA sequence of a lentiviral rev gene, an RNA sequence of a lentiviral vif gene, an RNA sequence of a lentiviral vpr gene, an RNA sequence of a lentiviral vpu gene, an RNA sequence of a lentiviral nef gene, and a U3 at the 3′ side of (c); the endogenous R region or heterologous polyadenylation (pA) signal at the 3′ side of the downstream U3, an internal ribosome entry site (IRES), a coding sequence encoding a protein, or one or more RNA sequences, each of which is complementary to either strand of a cloning site (such as a multiple cloning site) or a recombineering site.

3. The RNA of claim 2, wherein the pA signal is selected from the group of a simian virus 40 (SV40) pA signal, a bovine growth hormone (BGH) pA signal, or a thymidine kinase (TK) pA signal.

4. The RNA of any one of claims 1-3, wherein the SOI is heterologous to one or more of the other components of the RNA.

5. The RNA of any one of claims 1-4, wherein the SOI optionally comprises a micro RNA, a small interfering RNA (siRNA), a messenger RNA (mRNA), any other heterologous polynucleotide or wherein the SOI comprises an antisense strand of a donor template SUBSTITUTE SHEET (RULE 26) polynucleotide or an antisense oligonucleotide (ASO), optionally wherein the donor template polynucleotide serves as a template in the process of homologous recombination and that carries the modification that is to be introduced into a target sequence in a cell, further optionally thereby correcting one or more pathological mutations to its non-pathological wildtype nucleotide residue(s), and optionally wherein the micro RNA, or siRNA, or ASO interferes with a pathological gene expression and/or a pathological RNA (such as a pathological mRNA), and optionally wherein the SOI polynucleotide acts in adjuvantic fashion.

6. The RNA of any one or claim 1-5, wherein the SOI comprises one or more RNA sequence(s), each of which is complementary to either strand of a cloning site, or a multiple cloning site (MCS), or a recombineering site.

7. The RNA of any one of claims 1-6, comprising the following components, from 5′ to 3′:
(a) a SOI,
(b) a U5,
(c) a PBS,
(d) a Psi,
(e) an optional IRES,
(f) an optional coding sequence encoding a protein, or one or more RNA sequences, each of which is complementary to either strand of a cloning site or a recombineering site,
(g) a PPT,
(h) a U3, and
(i) the endogenous R or an optional pA signal.

8. The RNA of any one of claims 1-7, comprising a coding sequence encoding a protein, and wherein the protein encoded by the coding sequence is a clustered regularly interspaced short palindromic repeats (CRISPR) associated (Cas) enzyme and the SOI comprises a sense or antisense strand of a donor template polynucleotide suitable for use in a CRISPR system.

9. The RNA of claim 7, wherein the Cas enzyme is selected from: Cash, Cas9, Cas12a (Cpf1), Cas13, or a variant of each thereof.

10. The RNA of any one of claims 1-9, wherein the SOI is about 10 nucleotides (nt) long to about 10⁶ nt.

11. The RNA of any one of claims 1-10, wherein the SOI is no more than about 10⁵ nt.

12. The RNA of any one of claims 1-11, wherein the SOI is no more than about 10⁴ nt, optionally wherein the SOI is about 50 nt long, or about 500 nt long, or about 5000 nt long.

13. The RNA of any one of claims 1-12, wherein any of the non-SOI components is from or derived from a virus in the Retroviridae family.

14. The RNA of claim 13, wherein the virus is in the Orthoretrovirinae subfamily or the Spumaretrovirinae subfamily.

15. The RNA of claim 13 or 14, wherein the virus is selected from Alpharetrovirus, Betaretrovirus, Deltaretrovirus, Epsilonretrovirus, Gammaretrovirus, or Lentivirus.

16. The RNA of claim 15,
wherein the Alpharetrovirus is selected from Avian carcinoma Mill Hill virus 2, Avian leukosis virus (ALV), Avian myeloblastosis virus, Avian myelocytomatosis virus 29, Avian sarcoma leukosis virus (ASLV), Avian sarcoma virus CT10, Fujinami sarcoma virus, Rous sarcoma virus, UR2 sarcoma virus, or Y73 sarcoma virus;
wherein the Betaretrovirus is selected from Langur virus, Mason-Pfizer monkey virus (MPMV), Mouse mammary tumor virus (MMTV), Squirrel monkey retrovirus, or Jaagsiekte sheep retrovirus;
wherein the Deltaretrovirus is selected from Human T-lymphotropic virus (also named Human T-cell Leukaemia Virus, optionally selected from HTLV-1, HTLV-2, HTLV-3, HTLV-4), adult T-cell leukemia virus (ATLV), Simian-T-lymphotropic virus (types 1-4), Primate T-lymphotropic virus 1, Primate T-lymphotropic virus 2, Primate T-lymphotropic virus 3, or Bovine leukemia virus (BLV);
wherein the Epsilonretrovirus is selected from Walleye dermal sarcoma virus, Walleye epidermal hyperplasia virus 1, or Walleye epidermal hyperplasia virus 2;
wherein the Gammaretrovirus is selected from Chick syncytial virus, Murine Sarcoma Virus (MSV), Finkel-Biskis-Jinkins murine sarcoma virus, Gardner-Arnstein feline, sarcoma virus, Gibbon ape leukemia virus, Guinea pig type-C oncovirus, Hardy-Zuckerman, feline sarcoma virus, Harvey murine sarcoma virus, Kirsten murine sarcoma virus, Moloney murine sarcoma virus, Porcine type-C oncovirus, Reticuloendotheliosis virus, Snyder-Theilen feline sarcoma virus, Trager duck spleen necrosis virus, Viper retrovirus, Woolly monkey sarcoma virus, Murine leukemia virus (MLV), Abelson murine leukemia virus, Friend virus, Feline leukemia virus (FELV), Koala retrovirus (KoRV), or Xenotropic murine leukemia virus-related virus; and
wherein the Lentivirus is selected from human immunodeficiency virus (HIV), human immunodeficiency virus 1, human immunodeficiency virus 2, Simian immunodeficiency virus (SIV), Feline immunodeficiency virus (FIV), Puma lentivirus (PLV), Equine infectious anemia virus (EIAV), Bovine immunodeficiency virus (BIV), Caprine arthritis encephalitis virus, Jembrana disease virus, or Visna-maedi virus.

17. The RNA of claim 13 or 14, wherein the virus is selected from Bovispumavirus, Equispumavirus, Felispumavirus, Prosimiispumavirus, or Simiispumavirus.

18. The RNA of claim 17, wherein the virus is selected from Simian foamy virus or Human foamy virus.

19. The RNA of any one of claims 1-18, lacking a R region.

20. The RNA of any one of claim 1-19, wherein the RNA lacks a 5′ R region or a 3′ R region or both.

21. The RNA of any one of claims 1-20, wherein the RNA lacks a segment of the RNA that is located at the 5′ side of the U5 of (b).

22. The RNA of claim 21, wherein the segment is about 1 nt long to about 99 nt long.

23. The RNA of any preceding claim, further comprising a detectable or selection marker.

24. A polynucleotide complementary to or corresponding to the RNA of any one of claims 1-23 that optionally further comprises a detectable or selection marker.

25. The polynucleotide of claim 24, which is selected from the group consisting of a single-strand DNA, a single-strand RNA, or a single-strand polynucleotide comprising both deoxyribonucleotide residue(s) and ribonucleotide residue(s).

26. A polynucleotide encoding the RNA of any one of claims 1-23 that optionally further comprises a detectable or selection marker.

27. The polynucleotide of claim 26, further comprising a regulatory sequence that controls the transcription to the RNA.

28. The polynucleotide of claim 27, wherein the regulatory sequence comprises one or more of the sequences selected from the group consisting of: a promoter, a U3, an enhancer, an intron, a TATA box, an insulator, a silencer, 5′ cap, a polyadenylation sequence encoding a pA signal, a sequence encoding an IRES, a Woodchuck Hepatitis Virus (WHP) Posttranscriptional Regulatory Element (WPRE), or a signal sequence.

29. The polynucleotide of claim 28, wherein the promoter is a promoter (Pro) heterologous to one or more of the other components of the polynucleotide or any non-SOI component of the RNA encoded by the polynucleotide.

30. The polynucleotide of claim 28 or 29, wherein the promoter is suitable for use in an eukaryotic cell.

31. The polynucleotide of any one of claims 28-30, wherein the promoter is selected from the group of a cytomegalovirus immediate-early promoter (CMV), a simian virus 40 early promoter (SV40), or a Rous sarcoma virus LTR promoter (RSV).

32. A polynucleotide complementary to or corresponding to the polynucleotide of any one of claims 26-31.

33. A polynucleotide comprising one or more of the following: the RNA of any one of claims 1-23, or the polynucleotide of any one of claims 24-32.

34. A polynucleotide of any one of claims 26-31 which is selected from the group consisting of a single-strand DNA, a single-strand RNA, a single-strand polynucleotide comprising both deoxyribonucleotide residue(s) and ribonucleotide residue(s), a double-strand DNA, a double-strand RNA, a DNA/RNA hybrid, or any combination thereof.

35. A vector comprising one or more of the following: an RNA of any one of claims 1-23, or a polynucleotide of any one of claims 1-34.

36. The vector of claim 35, wherein the vector is a non-viral vector, optionally selected from plasmids, inorganic particles, calcium phosphate particles, silica nanoparticles, gold nanoparticles, nanoparticles, cationic lipids, lipid nano emulsions, solid lipid nanoparticles, peptide based vectors, polymer based vectors, liposomes, or gelatin-based vectors.

37. The vector of claim 35, wherein the vector is a viral vector, optionally selected from the group of a retroviral vector, a lentiviral vector, an adenoviral vector, or an adeno-associated viral vector.

38. A viral particle comprising an RNA of any one of claims 1-23.

39. The viral particle of claim 38, wherein the viral particle is in the Retroviridae family, and optionally in the Orthoretrovirinae subfamily or the Spumaretrovirinae subfamily.

40. The viral particle of claim 38 or 39, wherein the viral particle is selected from Alpharetrovirus, Betaretrovirus, Deltaretrovirus, Epsilonretrovirus, Gammaretrovirus, or Lentivirus.

41. The viral particle of claim 40,
wherein the Alpharetrovirus is selected from Avian carcinoma Mill Hill virus 2, Avian leukosis virus (ALV), Avian myeloblastosis virus, Avian myelocytomatosis virus 29, Avian sarcoma leukosis virus (ASLV), Avian sarcoma virus CT10, Fujinami sarcoma virus, Rous sarcoma virus, UR2 sarcoma virus, or Y73 sarcoma virus;
wherein the Betaretrovirus is selected from Langur virus, Mason-Pfizer monkey virus (MPMV), Mouse mammary tumor virus (MMTV), Squirrel monkey retrovirus, or Jaagsiekte sheep retrovirus;
wherein the Deltaretrovirus is selected from Human T-lymphotropic virus (also named Human T-cell Leukaemia Virus, optionally selected from HTLV-1, HTLV-2, HTLV-3, HTLV-4), adult T-cell leukemia virus (ATLV), Simian-T-lymphotropic virus (types 1-4), Primate T-lymphotropic virus 1, Primate T-lymphotropic virus 2, Primate T-lymphotropic virus 3, or Bovine leukemia virus (BLV);
wherein the Epsilonretrovirus is selected from Walleye dermal sarcoma virus, Walleye epidermal hyperplasia virus 1, or Walleye epidermal hyperplasia virus 2; wherein the Gammaretrovirus is selected from Chick syncytial virus, Murine Sarcoma Virus (MSV), Finkel-Biskis-Jinkins murine sarcoma virus, Gardner-Arnstein feline, sarcoma virus, Gibbon ape leukemia virus, Guinea pig type-C oncovirus, Hardy-Zuckerman, feline sarcoma virus, Harvey murine sarcoma virus, Kirsten murine sarcoma virus, Moloney murine sarcoma virus, Porcine type-C oncovirus, Reticuloendotheliosis virus, Snyder-Theilen feline sarcoma virus, Trager duck spleen necrosis virus, Viper retrovirus, Woolly monkey sarcoma virus, Murine leukemia virus (MLV), Abelson murine leukemia virus, Friend virus, Feline leukemia virus (FELV), Koala retrovirus (KoRV), or Xenotropic murine leukemia virus-related virus; and
wherein the Lentivirus is selected from human immunodeficiency virus (HIV), human immunodeficiency virus 1, human immunodeficiency virus 2, Simian immunodeficiency virus (SIV), Feline immunodeficiency virus (FIV), Puma lentivirus (PLV), Equine infectious anemia virus (EIAV), Bovine immunodeficiency virus (BIV), Caprine arthritis encephalitis virus, Jembrana disease virus, or Visna-maedi virus.

42. The viral particle of claim 38 or 39, wherein the viral particle is selected from Bovispumavirus, Equispumavirus, Felispumavirus, Prosimiispumavirus, or Simiispumavirus.

43. The viral particle of claim 42, wherein the viral particle is selected from Simian foamy virus or Human foamy virus.

44. A lentiviral particle comprising an RNA of any one of claims 1-23.

45. The particle of any one of claims 38-44, further comprising one or more of the following:
a protein encoded by a gag gene, a protein encoded by a pol gene, and a protein encoded by an env gene, optionally wherein the protein encoded by a gag gene is one or more of a group-specific antigen precursor polyprotein or its processed group-specific antigen polyprotein(s) selected from a nucleocapsid (NC), a capsid protein (CA) or a matrix protein (MA)), optionally wherein the protein encoded by a pol gene is one or more of as a precursor polyprotein encoded by a pol gene or its processed polyprotein(s) selected from a reverse transcriptase (RT), an RNase H domain optionally as part of a RT or any other polypeptide, an integrase (IN), or a protease (PR), optionally wherein the protein encoded by an env gene is one or more of a precursor polyprotein encoded by an env gene, or its processed polyprotein(s) selected from a surface envelope protein and a transmembrane envelope protein.

46. The particle of any one of claims 38-45, further comprising an endonuclease or a polynucleotide encoding an endonuclease.

46. The particle of any one of claims 38-45, further comprising a detectable or selection marker.

47. The particle of any one of claims 38-46, further comprising a lipid bilayer, optionally wherein the lipid bilayer further comprises the protein(s) encoded by an env gene, further optionally wherein the protein(s) encoded by an env gene is amphotropic, or ecotropic, or xenotropic, and yet further optionally wherein the protein(s) encoded by an env gene is derived from 10A1 MuLV envelopes, GaLV envelopes, VSV-G envelopes, or FeLVB envelopes.

48. The particle of any one of claims 38-47, wherein the RNase H is a wild type RNase H.

49. The particle of any one of claims 38-47, wherein the RNase H comprises a mutated RNase H defective in degrading RNA.

50. The particle of any one of claims 38-47 and 49, wherein the RNase H comprises one or more of Y586F, D524N, Δ5E, ΔC, or H7 mutations in a gammaretrovirus (RV) RNase H, or a E478Q mutation in a lentivirus (LV) RNase H domain.

51. The particle of any one of claims 38-47 and 49-50, wherein the RT is a wild type RT, or wherein the RT comprises a mutated RT defective in mediating strand transfer, or wherein the RT is a High Fidelity Reverse Transcriptase, and optionally wherein the High Fidelity Reverse Transcriptase is a mutant LV RT comprising the mutations of W229A and V751 and K65R.

52. The particle of any one of claims 38-47 and 49-51, wherein the RT comprises a L92P mutant and/or a F61A of a lentivirus (LV) RT or a Y598V mutant of a retrovirus (RV) RT.

53. The particle of any one of claims 38-52, wherein the NC is a wildtype NC.

54. The particle of any one of claims 38-52, wherein the NC is a mutated NC defective in mediating strand transfer.

55. The particle of any one of claims 38-54, wherein the integrase is a wildtype integrase.

56. The particle of any one of claims 38-54, wherein the integrase is defective in integrating a polynucleotide into a chromosomal DNA, and optionally wherein the integrase comprises a D64V mutation.

57. The particle of any one of claims 38-45, 47, 52-54, and 56, comprising the following:
a vector genome comprising an RNA of any one of claims 1-23,
a capsid comprising a capsid protein (CA) encoded by a gag gene and a matrix protein (MA) encoded by a gag gene,
a lipid bilayer further comprising an envelope protein encoded by an env gene,
an RT encoded by a pol gene, wherein the RT is a L92P mutant and/or a F61A of a lentivirus (LV) RT or a Y598V mutant of a retrovirus (RV) RT,
a nucleocapsid (NC), and
an optional integrase that is defective in integrating a polynucleotide into a chromosomal DNA.

58. The particle of any one of claims 38-45, 47, 50, 53-54, and 56, comprising the following:
a vector genome comprising an RNA of any one of claims 1-23,
a capsid comprising a capsid protein (CA) encoded by a gag gene and a matrix protein (MA) encoded by a gag gene,
a lipid bilayer further comprising an envelope protein encoded by an env gene,
an RNase H encoded by a pol gene, wherein the RNase H comprises one or more of Y586F, D524N, Δ5E, ΔC, or H7 mutations in a gammaretrovirus (RV) RNase H, or a E478Q mutation in a lentivirus (LV) RNase H domain,
a nucleocapsid (NC), and
an optional integrase that is defective in integrating a polynucleotide into a chromosomal DNA.

59. The particle of any one of claims 38-45, 47-52, 54, and 56, comprising the following:
a vector genome comprising the engineered RNA of any one of claims 1-12,
a capsid comprising a capsid protein encoded by a gag gene and a matrix protein (MA) encoded by a gag gene,
a lipid bilayer further comprising an envelope protein encoded by an env gene, an RNase H encoded by a pol gene,
a nucleocapsid (NC), wherein the NC is a mutated NC defective in mediating strand transfer, and
an optional integrase that is defective in integrating a polynucleotide into a chromosomal DNA.

60. A cell comprising one or more of the following: an RNA of any one of claims 1-23, a polynucleotide of any one of claims 24-34, a vector of any one of claims 35-37, or a particle of any one of claims 38-59.

61. The cell of claim 60, wherein the cell is derived from a packaging cell line optionally selected from the group consisting of psi-2, psi-Crypt, psi-AM, GP+E-86, PA317, GP+envAM-12, Fly A13, BOSC 23, BING, Fly RD 18, ProPak-X, -A.52 and -A.6.

62. The cell of claim 60 or 61, further comprising or expressing one or more of the following: a protein encoded by a gag gene, a protein encoded by a pol gene, a protein encoded by an env gene, a protein encoded by a lentiviral tat gene, a protein encoded by a lentiviral rev gene, a protein encoded by a lentiviral vif gene, a protein encoded by a lentiviral vpr gene, a protein encoded by a lentiviral vpu gene, a protein encoded by a lentiviral nef gene, a gag gene, a pol gene, an env gene, a lentiviral tat gene, a lentiviral rev gene, a lentiviral vif gene, a lentiviral vpr gene, a lentiviral vpu gene, or a lentiviral nef gene, optionally wherein the protein encoded by a gag gene is one or more of a group-specific antigen precursor polyprotein or its processed group-specific antigen polyprotein(s) selected from a nucleocapsid (NC), a capsid protein (CA) or a matrix protein (MA)), optionally wherein the protein encoded by a pol gene is one or more of as a precursor polyprotein encoded by a pol gene or its processed polyprotein(s) selected from a reverse transcriptase (RT), an RNase H domain optionally as part of a RT or any other polypeptide, an integrase (IN), or a protease (PR), and optionally wherein the protein encoded by an env gene is one or more of a precursor polyprotein encoded by an env gene, or its processed polyprotein(s) selected from a surface envelope protein and a transmembrane envelope protein, further optionally wherein the protein(s) encoded by an env gene is amphotropic, or ecotropic, or xenotropic, and yet further optionally wherein the protein(s) encoded by an env gene is derived from 10A1 MuLV envelopes, GaLV envelopes, VSV-G envelopes, or FeLVB envelopes.

63. The cell of any one of claims 60-62, further comprising one or more of the following: an endonuclease, a polynucleotide encoding an endonuclease, or a polynucleotide that is a reverse complement of the endonuclease-coding polynucleotide.

64. The cell of claim 62 or 63, wherein the RNase H is a wildtype RNase H.

65. The cell of claim 62 or 63, wherein the RNase H comprises a mutated RNase H defective in degrading RNA.

66. The cell of any one of claims 62-63 and 65, wherein the RNase H comprises one or more of Y586F, D524N, Δ5E, ΔC, or H7 mutations in a gammaretrovirus (RV) RNase H or a E478Q mutation in a lentivirus (LV) RNase H domain.

67. The cell of any one of claims 62-63 and 65-66, wherein the RT is a wild type RT, or wherein the RT comprises a mutated RT defective in mediating strand transfer, or wherein the RT is a High Fidelity Reverse Transcriptase, and optionally wherein the High Fidelity Reverse Transcriptase is a mutant LV RT comprising the mutations of W229A and V751 and K65R.

68. The cell of any one of claims 62-63 and 65-67, wherein the RT comprises a F61A and/or L92P mutant of a lentivirus (LV) RT or a Y598V mutant of a retrovirus (RV) RT.

69. The cell of any one of claims 62-68, wherein the NC is a wildtype NC.

70. The cell of any one of claims 62-68, wherein the NC is a mutated NC defective in mediating strand transfer.

71. The cell of any one of claims 62-70, wherein the integrase is a wildtype integrase.

72. The cell of any one of claims 62-70, wherein the integrase is defective in integrating a polynucleotide into a chromosomal DNA.

73. The cell of any one of claims 60-72, comprising any one or any two or all three of the following:
(a) an expression vector comprising a gag gene and a pol gene,
(b) an expression vector comprising an env gene, or
(c) a polynucleotide of any one of claim 26-31 or 33-34.

74. The cell of any one of claims 60-73, comprising any one or any two or all three of the following:
(a) an expression vector comprising a gag gene and a pol gene,
(b) an expression vector comprising an env gene, or
(c) a vector of any one of claims 35-37.

75. A method of producing a retroviral or lentiviral particle, comprising (a) culturing a cell comprising one or more of the following into a cell at least one of: an RNA of any one of claims 1-23, a polynucleotide of any one of claims 24-34, or a vector of any one of claims 35-37, wherein the cell comprises or expresses one or more of the following: a protein encoded by a gag gene, a protein encoded by a pol gene, a protein encoded by an env gene, a protein encoded by a lentiviral tat gene, a protein encoded by a lentiviral rev gene, a protein encoded by a lentiviral vif gene, a protein encoded by a lentiviral vpr gene, a protein encoded by a lentiviral vpu gene, a protein encoded by a lentiviral nef gene, a protein encoded by another retroviral or lentiviral accessory gene(s), a gag gene, a pol gene, an env gene, a lentiviral tat gene, a lentiviral rev gene, a lentiviral vif gene, a lentiviral vpr gene, a lentiviral vpu gene, a lentiviral nef gene, or another retroviral or lentiviral accessory gene(s), optionally wherein the protein encoded by a gag gene is one or more of a group-specific antigen precursor polyprotein or its processed group-specific antigen polyprotein(s) selected from a nucleocapsid (NC), a capsid protein (CA) or a matrix protein (MA)), optionally wherein the protein encoded by a pol gene is one or more of as a precursor polyprotein encoded by a pol gene or its processed polyprotein(s) selected from a reverse transcriptase (RT), an RNase H domain optionally as part of a RT or any other polypeptide, an integrase (IN), or a protease (PR), and optionally wherein the protein encoded by an env gene is one or more of a precursor polyprotein encoded by an env gene, or its processed polyprotein(s) selected from a surface envelope protein and a transmembrane envelope protein;
(b) optionally collecting supernatant of the cell culture; and
(c) optionally isolating or purifying retroviral or lentiviral particles from the collected supernatant;

76. A method of producing a retroviral or lentiviral particle, comprising (a) culturing a cell of any one of claims 38-49, wherein the cell comprise or expresses one or more of the following: one or more of the following: a protein encoded by a gag gene, a protein encoded by a pol gene, a protein encoded by an env gene, a protein encoded by a lentiviral tat gene, a protein encoded by a lentiviral rev gene, a protein encoded by a lentiviral vif gene, a protein encoded by a lentiviral vpr gene, a protein encoded by a lentiviral vpu gene, a protein encoded by a lentiviral nef gene, a protein encoded by another retroviral or lentiviral accessory gene(s), a gag gene, a pol gene, an env gene, a lentiviral tat gene, a lentiviral rev gene, a lentiviral vif gene, a lentiviral vpr gene, a lentiviral vpu gene, a lentiviral nef gene, or another retroviral or lentiviral accessory gene(s), optionally wherein the protein encoded by a gag gene is one or more of a group-specific antigen precursor polyprotein or its processed group-specific antigen polyprotein(s) selected from a nucleocapsid (NC), a capsid protein (CA) or a matrix protein (MA)), optionally wherein the protein encoded by a pol gene is one or more of as a precursor polyprotein encoded by a pol gene or its processed polyprotein(s) selected from a reverse transcriptase (RT), an RNase H domain optionally as part of a RT or any other polypeptide, an integrase (IN), or a protease (PR), and optionally wherein the protein encoded by an env gene is one or more of a precursor polyprotein encoded by an env gene, or its processed polyprotein(s) selected from a surface envelope protein and a transmembrane envelope protein;
(b) optionally collecting supernatant of the cell culture; and
(c) optionally isolating or purifying retroviral or lentiviral particles from the collected supernatant.

77. The method of claim 75 or 76, wherein the cell is cultured for about 48 hours to about 72 hours.

78. The method of any one of claims 75-77, further comprising optionally concentrating the isolated or purified retroviral or lentiviral particles.

79. The method of any one of claims 75-78, wherein the cell further expresses an endonuclease.

80. The method of any one of claims 75-79, wherein the cell further comprises a polynucleotide encoding an endonuclease or a polynucleotide that is a reverse complement of the endonuclease-coding polynucleotide.

81. A retroviral or lentiviral particle produced by the method of any one of claims 75-80.

82. A method of delivering a polynucleotide to a cell, comprising contacting the cell with a particle of any one of claim 38-59 or 81, wherein the delivered polynucleotide comprises the sequence of interest (SOI) or a polynucleotide which is a reverse complement of the SOI or both.

83. The method of claim 82, wherein the cell is a eukaryotic cell or a prokaryotic cell.

84. A method of delivering a polynucleotide to a subject, comprising administering a particle of any one of claim 38-59 or 81 to the subject, wherein the delivered polynucleotide comprises the sequence of interest (SOI) or a polynucleotide which is a reverse complement of the SOI or both.

85. The method of claim 84, wherein the subject is a mammal.

86. A method of producing a polynucleotide, comprising
(a) contacting a cell with a particle of any one of claim 38-59 or 81,
(b) culturing the cell, and
(c) optionally isolating or purifying the polynucleotide produced by the cell, wherein the produced polynucleotide comprises the sequence of interest (SOI) or a polynucleotide that is a reverse complement of the SOI or both.

87. The method of claim 86, further comprising optionally concentrating the isolated or purified polynucleotide.

88. The method of any one of claims 82-87, wherein the delivered or produced polynucleotide is not a double-stranded DNA.

89. The method of any one of claims 82-88, wherein the delivered or produced polynucleotide is a single-stranded DNA or a RNA-DNA hybrid.

90. The method of any one of claims 82-89, wherein the delivered or produced polynucleotide is free of any retroviral or lentiviral sequence at the 5′ end or the 3′ end or both ends.

91. The method of any one of claims 82-90, wherein the delivered or produced polynucleotide comprises one or more of the following: a donor template polynucleotide, a micro RNA, a small interfering RNA (siRNA), a messenger RNA (mRNA), an antisense oligonucleotide (ASO), or any heterologous polynucleotide.

92. The method of any one of claims 82-91, wherein the particle comprises an endonuclease or a polynucleotide encoding an endonuclease, and wherein the endonuclease cleaves the delivered or produced polynucleotide and releases one or more of the following: a donor template polynucleotide, a micro RNA, a small interfering RNA (siRNA), a messenger RNA (mRNA), an antisense oligonucleotide (ASO), a DNA-RNA hybrid, or any heterologous polynucleotide.

93. The method of any one of claims 82-85 and 88-92, further comprising delivering a guide polynucleotide to the cell or subject.

94. The method of claim 93, wherein the guide polynucleotide is a guide RNA (gRNA) suitable for use in a CRISPR system.

95. The method of claim 93 or 94, wherein the guide polynucleotide is delivered in a non-viral or viral vector.

96. A polynucleotide delivered to a cell or a subject using the method of any one of claims 82-85 and 88-95.

97. A polynucleotide produced by the method of any one of claims 82-95.

98. A composition comprising a particle of any one of claim 38-59 or 81, and a carrier.

99. The composition of claim 98, wherein the carrier is a pharmaceutically acceptable carrier.

100. The composition of claim 98 or 99, further comprising a preservative or stabilizer.

101. The composition of any one of claims 98-100, further comprising a guide polynucleotide.

102. The composition of claim 101, wherein the guide polynucleotide is a guide RNA (gRNA) suitable for use in a CRISPR system.

103. The composition of claim 101 or 102, wherein the guide polynucleotide is in a non-viral or viral vector.

104. A kit comprising one or more of the following: an RNA of any one of claims 1-23; a polynucleotide of any one of claim 24-34 or 96-97; a vector of any one of claims 35-37; a particle of any one of claim 38-59 or 81; a cell of any one of claims 60-74; an expression vector comprising one or more of: a gag gene, a pol gene, an env gene, a lentiviral tat gene, a lentiviral rev gene, a lentiviral vif gene, a lentiviral vpr gene, a lentiviral vpu gene, or a lentiviral nef gene; an expression vector encoding proteins required for the RNA to be packaged in a particle; the composition of any one of claims 98-103, and an optional instruction for use.

105. A provirus comprising one or more of the following: an RNA of any one of claims 1-23, or a polynucleotide of any one of claim 24-34 or 96-97.

106. A messenger RNA (mRNA) of the provirus of claim 105.
